(19) United States
(12) Patent Application Publication
(10) Pub. No.: US 2010/0093090 A1
(43) Pub. Date: Apr. 15, 2010

(54) METHOD AND KIT FOR EFFICIENT REPROGRAMMING OF SOMATIC CELLS

(75) Inventors: HONGKUI DENG, BEIJING (CN); YANG ZHAO, BEIJING (CN); XIAOLEI YIN, BEIJING (CN); QIANG ZHANG, BEIJING (CN); FANGFANG ZHU, BEIJING (CN); HAISONG LIU, BEIJING (CN); WEIFENG YANG, BEIJING (CN); CHENGANG XIANG, BEIJING (CN); HAN QIN, BEIJING (CN)

Correspondence Address: KNOBBE MARTENS OLSON & BEAR LLP, 2040 MAIN STREET, FOURTEENTH FLOOR, IRVINE, CA, 92614, US

(73) Assignees: PEKING UNIVERSITY, BEIJING (CN); BEIJING HUA YUAN BO CHUANG TECHNOLOGY CO., LTD., BEIJING (CN)

(21) Appl. No.: 12/568,634

(22) Filed: Sep. 28, 2009

(30) Foreign Application Priority Data
Apr. 3, 2008 (CN) 200810091841.2

(51) Int. Cl. C12N 5/071; C12N 5/00

(52) U.S. Cl. 435/377; 435/325

(57) ABSTRACT

The present invention relates to method or kit for efficient reprogramming of somatic cells.

### RELATED APPLICATIONS

This application claims the benefit of Chinese Patent Application No. 200810091841.2, filed Apr. 3, 2008, which is hereby expressly incorporated by reference in its entirety.

### FIELD OF THE INVENTION

The invention relates to the conversion of a differentiated somatic cell into a naive cell, e.g., stem cell such as an induced pluripotent stem cell.

### DESCRIPTION OF THE RELATED ART

It is reported that a somatic cell could be reprogrammed to be a stem cell, which is more naive and have more potential of self-renewal and differentiation. This reprogramming process could be induced by nuclear transplantation into Oocytes (Campbell, McWhir et al. 1996; Jouneau and Renard 2003), by fusion with pluripotent stem cells (Cowan, Atienza et al. 2005), by contacting with chemical compounds, or induced by culture methods (Chen, Zhang et al. 2004).

In August, 2006, Yamanaka's lab reported that mouse embryonic fibroblast cells could be reprogrammed to be “induced pluripotent stem cells (iPS cells)”, through retro-viral introduction of four transcriptional factors, Oct4, Sox2, Klf4 and c-Myc (Takahashi and Yamanaka 2006). After that, Oct4, Sox2, Klf4 and c-Myc were so-called reprogramming factors. In November 2007, Yamanaka's lab and Thomson's lab both reported the generation of human iPS cells from adult human fibroblasts by the combination “Oct4, Sox2, Klf4 and c-Myc” or “Oct4, Sox2, Nanog, Lin-28”, respectively (Takahashi, Tanabe et al. 2007; Yu, Vodyanik et al. 2007). IPS cells were very similar to embryonic stem cells in gene profiling, differentiation potential and epigenetic modifications (Maherali, Sridharan et al. 2007). They were able to self-renew and differentiate into all mature cell types, including neurons, hematopoietic cells, muscle cells and islet cells.

Further study found that c-myc was not necessary in this combination. iPS cells could be induced by Oct4, Sox2 and Klf4, without c-myc although the efficiency was much lower (Nakagawa, Koyanagi et al. 2008; Wernig, Meissner et al. 2008). Further study showed that, Sox2, Klf4 and c-myc could also be substituted by other members in their gene family. For examples, Sox2 could be substituted by Sox1 or Sox3; Klf4 could be substituted by KLF2 and KLF5; while, c-myc could be substituted by L-myc and N-myc (Nakagawa, Koyanagi et al. 2008). Other genes were identified to improve the reprogramming efficiency or replace some of the four transcriptional factors in this combination. For example, Esrrb could substitute Klf4 to induce iPS cells with Oct4 and Sox2 (Feng, Jiang et al. 2009); SV40 T could greatly enhance reprogramming efficiency (Mali, Ye et al. 2008). We found that p53 inactivation factor and UTF1 could greatly improve iPS cell generation efficiency (Zhao, Yin et al. 2008). After that, six groups reported that p53 pathway was a major barrier of iPS cell generation (Banito, Rashid et al. 2009; Hong, Takahashi et al. 2009; Kawamura, Suzuki et al. 2009; Li, Collado et al. 2009; Marion, Strati et al. 2009; Utikal, Polo et al. 2009).

Particularly, several chemical compounds were reported to be involved in reprogramming process. VPA (an HDAC inhibitor) and 5-aza-C (a DNA methyl transferase inhibitor) were reported to improve reprogramming efficiency (Huangfu, Maehr et al. 2008; Mikkelsen, Hanna et al. 2008). CHIR 99021 (a GSK3 inhibitor) and PD0325901 (an MEK/ERK inhibitor) could help push partial reprogrammed cells into fully reprogrammed iPS cells (Silva, Barrandon et al. 2008). Furthermore, BIX01294 were reported to replace oct4 to induce iPS cells from NSCs (Shi, Do et al. 2008). VPA were also reported to replace KLF4 and induce reprogramming with only Oct4 and Sox2 from human foreskin fibroblasts (Huangfu, Osafune et al. 2008). BIX01294 and BAYK 8644 were reported to induce iPS cells with only Oct4 and Klf4 (Shi, Desponts et al. 2008). Kenpaullone could substitute KLF4 and induce iPS cells with Oct4, sox2 and c-myc (Lyssiotis, Foreman et al. 2009).

There were cell types more amenable to reprogramming. For example, human keratinocytes and amniotic fluid-derived cells were reported to be reprogrammed by a much higher efficiency (Aasen, Raya et al. 2008; Li, Zhou et al. 2009). NSCs were similar to ESCs (or iPSCs) in gene profiling, and express Sox2, c-myc and Klf4. They could be induced into iPS cells with only one transcriptional factor, Oct4, both in mouse and human (Kim, Greber et al. 2009; Kim, Sebastiano et al. 2009). Melanocytes were also reported to be induced to iPS cells without sox2, although there was no evidence to their expression of sox2 (Utikal, Maherali et al. 2009). Moreover, somatic cells from patients were also reported to be induced to iPS cells for potential clinical applications (Dimos, Rodolfa et al. 2008; Park, Arora et al. 2008; Raya, Rodriguez-Piza et al. 2009).

Furthermore, iPS cells could be induced not limited to retro-viral infection. DNA transduction, adenoviral delivery or protein delivery of the four reprogramming factors could also be applied in iPS cell induction (Okita, Nakagawa et al. 2008; Stadtfeld, Nagaya et al. 2008; Kim, Kim et al. 2009; Zhou, Wu et al. 2009; Zhou and Freed 2009). Moreover, the four reprogramming factors could also be conjunct in a single polycistronic vector to induce iPS cells (Carey, Markoulaki et al. 2009; Sommer, Stadtfeld et al. 2009).

### SUMMARY OF THE INVENTION

The methods described herein can be used, for example, to optimize (e.g., improve speed or efficiency) the creation of induced cells, e.g., induced pluripotent stem (iPS) cells from other cell types (e.g., an adult cell and/or a somatic cell), including, but not limited to the creation of iPS cells from human biopsies, such as blood, skin, fat, hair follicle, mucus, etc. The iPS cell lines so created can be used to study differentiation and disease mechanisms/pathology.

In one aspect, the invention features a method for reprogramming a somatic cell, wherein the method comprises treating the somatic cell with factor A selected from one or more of the group consisting of Oct4, Klf4, Sox2, c-Myc, a MEK/ERK inhibitor, BIX01294, BAYK 8644, Kenpaullone, a GSK3 inhibitor, an HDAC inhibitor and a DNA methyl transferase inhibitor, and factor B selected from one or two of the group consisting of UTF1 and a p53 inactivation factor, or the combinations thereof.

In a preferable embodiment, the MEK/ERK inhibitor is PD0325901, the HDAC inhibitor is VPA, the GSK3 inhibitor is CHIR 99021, and the DNA methyl transferase inhibitor is 5-aza-C.

In one aspect, the invention features a method of reprogramming an iPS cell from a somatic cell, the method comprising: introducing factor A and factor B into a somatic cell under conditions sufficient to produce an iPS cell from a somatic cell, wherein factor A is selected from one or more (e.g. two, three, four, five, six, seven, eight, nine, or ten) of the group consisting of Oct4, Klf4, Sox2, c-Myc, a MEK/ERK inhibitor, BIX01294, BAYK 8644, Kenpaullone, a GSK3 inhibitor, an HDAC inhibitor and a DNA methyl transferase inhibitor, and factor B is selected from one or two of the group consisting of UTF1 and a p53 inactivation factor, or the combinations thereof.

In some embodiments, factor A selected from one or more of the group consisting of Oct4, Klf4, Sox2, and c-Myc and/or factor B are in the form of DNA, mRNA or proteins.

In some embodiments, the step of treating the somatic cell with factor A selected from one or more of the group consisting of Oct4, Klf4, Sox2 and c-Myc and factor B selected from one or two of the group consisting of UTF1 and a p53 inactivation factor comprises treating the somatic cell with one heterologous nucleic acid sequence encoding said factor A and said factor B, or the respective heterologous nucleic acid sequences encoding Oct4, Klf4, Sox2, c-Myc, UTF1 or a p53 inactivation factor, or the combinations thereof.

In some embodiments, the somatic cell is treated with the heterologous nucleic acid sequence by infection.

In some embodiments, a p53 inactivated factor is a double strand small RNA and the DNA sequence encoding this small interference RNA is GACTCCAGTGGTAATCTACT.

In some embodiments, a p53 inactivation factor selectively inhibits p53 gene expression.

In some embodiments, a p53 inactivation factor selectively inhibits p53 protein modification, and subsequently impairs p53 protein function.

In some embodiments, a p53 inactivation factor comprises at least two p53 siRNA, or a combination thereof.

In some embodiments, a p53 inactivation factor is a small molecule selectively inhibits p53 mediated signaling pathway. For example, KU55933, which is a p53 pathway inhibitor, which selectively and competitively inhibiting ATM kinase, p53 activity and p21 expression.

In some embodiments, the somatic cell is treated by CHIR 99021 or other chemical compounds reported to improve reprogramming efficiency. In a preferable embodiment, said chemical compounds reported to improve reprogramming efficiency is a MEK/ERK inhibitor, BIX01294, BAYK 8644, Kenpaullone, a GSK3 inhibitor, an HDAC inhibitor and a DNA methyl transferase inhibitor. In a preferable embodiment, the MEK/ERK inhibitor and the GSK3 inhibitor is as shown above, the DNA methyl transferase inhibitor is as shown below, and HDAC inhibitor is as shown below.

In some embodiments, the method comprises treating the somatic cell with two transcription factors.

In some embodiments, the transcription factors comprise Oct4 and Sox2.

In some embodiments, the method comprises treating the somatic cell with three transcription factors.

In some embodiments, the transcription factors comprise Oct4, Sox2 and Klf4.

In some embodiments, the method comprises treating the somatic cell with four transcription factors.

In some embodiments, the transcription factors comprise Oct4, Sox2, Klf4 and c-Myc.

In some embodiments, the somatic cell is reprogrammed partially or completely. In a preferable embodiment, the somatic cell is reprogrammed completely into the induced pluripotent stem cell.

In some embodiments, the iPS cell has a normal karyotype.

In some embodiments, the somatic cell is a fibroblast (e.g., a primary fibroblast), a muscle cell (e.g., a myocyte), a cumulus cell, a neural cell, a liver cell, a GI tract cell, a mammary cell, a hepatocyte, a stomach cell, a keratinocyte cell, a kidney cell, a blood cell, a vascular cells, a skin cell, an immune system cell, a lung cell, a bone cell or a pancreatic islet cell.

In some embodiments, the somatic cell is a primary cell or is a progeny of a primary or secondary cell.

In some embodiments, the somatic cell is derived from mammal. In a preferable embodiment, the mammal is human, murine, or primate.

In some embodiments, the somatic cell is obtained from a sample selected from a group consisting of a hair follicle, a blood sample, a swab sample or an adipose biopsy.

In some embodiments, the somatic cell is a healthy cell or a cell containing at least one genetic lesion.

In some embodiments, a plurality of the iPS cells are produced from a plurality of the somatic cells.

In some embodiments, the method further comprises implanting the iPS cells into a subject.

In some embodiments, the subject is suffering from a disorder. In a preferable embodiment, the disorder is selected from a group consisting of hematopoietic conditions (e.g., sickle cell anemia, leukemias, immune deficiencies), cardiac disorders (e.g., myocardial infarcts, and myopathies) and disorders such as liver disease, diabetes, thyroid abnormalities, neurodegenerative/neurological disorders (e.g., Parkinson's, Alzheimer's, stroke injuries, spinal chord injuries), circulatory disorders, respiratory disorders, wound healing and/or repair, bone repair, and enzyme abnormalities.

In some embodiments, the iPS cells are from a donor different than the subject (e.g., a relative of the subject).

In another aspect, the present invention features a method for improving the reprogramming efficiency of a somatic cell, the method comprising treating the somatic cell with factor B selected from one or two of the group consisting of UTF1 and a p53 inactivation factor.

In still another aspect, the present invention features a method for preparing a medicament for treating disease, characterized in that the usage of the cell produced by the method of claim 1, wherein the disease is selected from the group consisting of hematopoietic conditions, cardiac disorders and disorders such as liver disease, diabetes, thyroid abnormalities, neurodegenerative/neurological disorders, circulatory disorders, respiratory disorders, wound healing and/or repair, bone repair, and enzyme abnormalities.

In a preferable embodiment of all of the above aspects, the somatic cell is treated with a MEK/ERK inhibitor, BIX01294, BAYK 8644, Kenpaullone, a GSK3 inhibitor, an HDAC inhibitor and/or a DNA methyl transferase inhibitor before/after the treatment of the somatic cell with one or more of the factor selected from the group consisting of Oct4, Klf4, Sox2, c-Myc, UTF1 and a p53 inactivation factor.

In another aspect, the invention features an iPS cell produced by the method described herein.

In one aspect, the invention features a cell expressing Oct4, Sox2, Klf4, c-Myc and UTF1.

In another aspect, the invention features a cell expressing Oct4, Sox2, Klf4, c-Myc and expressing low p53.

In one aspect, the invention features a cell expressing Oct4, Sox2, Klf4 and UTF1.

In one aspect, the invention features a cell expressing Oct4, Sox2, Klf4, and low expressing p53.

In one aspect, the invention features a cell expressing Oct4, Sox2 and UTF1.

In one aspect, the invention features a cell expressing KLF4, and UTF1.

In one aspect, the invention features a cell expressing Oct4, Sox2, and low expressing p53.

In one aspect, the invention features a cell expressing KLF4, and low expressing p53.

In one aspect, the invention features a reaction mixture comprising a more primitive precursor or a less differentiated cell, e.g., a pluripotent stem cell (or a population thereof) compared to a somatic cell from which it was derived and expressed exogenous UTF1 and/or a p53 inactivation factor.

In some embodiments, the less differentiated cell is an iPS cell.

In some embodiments, the iPS cell is produced by a method described herein.

In one aspect, the invention features a composition comprising a cell produced by a method described herein.

In another aspect, the present invention features a kit for reprogramming a somatic cell, comprising factor A selected from one or more (for example, two, three, four, five, six, seven, eight, nine or ten) of the group consisting of Oct4, Klf4, Sox2, c-Myc, a MEK/ERK inhibitor, BIX01294, BAYK 8644, Kenpaullone, a GSK3 inhibitor, an HDAC inhibitor and a DNA methyl transferase inhibitor, as well as factor B selected from one or two of the group consisting of UTF1 and a p53 inactivation factor.

In a preferable embodiment, the MEK/ERK inhibitor is PD0325901, the HDAC inhibitor is VPA, the GSK3 inhibitor is CHIR 99021, and the DNA methyl transferase inhibitor is 5-aza-C.

In still another aspect, the present invention features a kit comprising: an iPS cell produced by a method of claim 1; at least one component for directing the iPS cell to a differentiated cell or expanding the iPS cell; and instructions.

In a preferable embodiment, the iPS cell is frozen or in culture.

In some embodiments, the p53 inactivated factor and the somatic cell are as described above. In some embodiments, the kit further comprises a component for the detection of a marker for an iPS cell selected from a group consisting of alkaline phophatase, NANOG, OCT4, SOX2, SSEA4, TRA-1-60 and TRA-1-81. Those skilled in the art can determine the component for the detection of a marker based on the knowledge in the art. In some embodiments, the expression of a marker selected from a group consisting of alkaline phophatase, NANOG, OCT4, SOX2, SSEA4, TRA-1-60 and TRA-1-81 is upregulated to by a statistically significant amount in the iPS cell relative to the somatic cell.

In some embodiments, the kit further comprises an iPS cell wherein the iPS cell is produced from the same cell type of the somatic cell by the method described herein.

In some embodiments, the kit further comprises a component for preparation of a karyotype from a cell.

In another aspect, the invention features a kit comprising an iPS cell produced by the method described herein. In a preferable embodiment, the p53 inactivated factor is as described above.

In some embodiments, the iPS cell is an isolated iPS cell.

In some embodiments, the iPS cell is frozen or in culture.

In yet another aspect, the invention features a kit comprising: an iPS cell produced by the method described herein; at least one component for directing the iPS cell to a differentiated cell; and instructions for directing the iPS cell to a differentiated cell.

In some embodiments, the iPS cell is an isolated iPS cell.

In some embodiments, the iPS cell is frozen or in culture.

In some embodiments, the differentiated cell comprises a fibroblast (e.g., primary fibroblast), a muscle cell (e.g., a myocyte), a cumulus cell, a neural cell, a liver cell, a GI tract cell, a mammary cell, a kidney cell, a blood cell, a vascular cell, a skin cell, an immune system cell, a lung cell, a bone cell, keratinocyte, or a pancreatic islet cell.

In one aspect, the invention features a kit comprising: an iPS cell produced by the method described herein; at least one component for expanding the iPS cell; and instructions for expanding the iPS cell.

In some embodiments, the iPS cell is an isolated iPS cell.

In some embodiments, the iPS cell is frozen or in culture.

In one aspect, the invention features a method of instructing an end-user to produce an iPS cell from a somatic cell, the method comprises providing the components of a kit for producing an iPS cell from a somatic cell described herein; and instructing the end-user using an information material, e.g., a printed material or a computer readable material, or both.

Accordingly, in one aspect, the disclosure features a method of producing a more primitive precursor or a less differentiated cell, e.g., pluripotent stem cell (or a population thereof) from a somatic cell, or reprogramming a somatic cell. The method comprises: introducing UTF1 or a p53 inactivation factor, or their combination thereof, thereby produce a primitive precursor or a less differentiated cell, e.g., pluripotent stem cell (or a population thereof) from the somatic cell or to reprogram the somatic cell.

In one embodiment of any one of above aspects, the somatic cell further expresses, or has increased expression, of one or more transcription factor(s) (e.g., two, three, or four transcription factors). In one embodiment, the transcription factor is one or more of Oct4, Klf4, Sox2 and c-Myc. In one embodiment, the somatic cell does not express c-Myc or does not express c-Myc at statistically significant levels or does not over express c-Myc.

In one embodiment of any one of above aspects, the somatic cell does not express c-Myc or Klf4 or does not express c-Myc or Klf4 at statistically significant levels or does not over express c-Myc and Klf4. In some embodiments, the somatic cell can express, e.g., Oct4 and Sox2 or the somatic cell can express, e.g, Oct4, Klf4 and Sox2 or the somatic cell can express Oct4, Klf4, Sox2 and c-Myc.

In one embodiment of any one of above aspects, the somatic cell includes a heterologous nucleic acid sequence, e.g., a heterologous nucleic acid sequence encoding transcription factor(s), e.g., a nucleic acid encoding transcription factor(s) described herein. In one embodiment, the nucleic acid encodes Oct4, Klf4, Sox2 or c-Myc. In one embodiment, the somatic cell includes one or more heterologous nucleic acid sequences, e.g., encoding transcription factors, e.g., encoding one or more of Oct4, Klf4, Sox2 and c-Myc. In one embodiment, the somatic cell includes at least three heterologous nucleic acid sequences, e.g., encoding Oct4, Klf4 and Sox2. In one embodiment, somatic cell includes at least two heterologous nucleic acid sequences, e.g., encoding Oct4 and Sox2. In another embodiment, the somatic cell includes at least four heterologous nucleic acid sequences, e.g., encoding Oct4, Klf4, Sox2 and c-Myc. In one embodiment, the nucleic acid sequence is introduced into the somatic cell, or the somatic cell is the progeny of such a somatic cell. In an embodiment the cell does not include a heterologous c-Myc gene. In an embodiment the cell does not include heterologous c-Myc and Klf4 genes. In an embodiment the somatic cell is human and any heterologous gene, e.g., transcription factor gene, is human as well, e.g., the human equivalent of any of Oct4, Klf4, Sox2 and c-Myc. In an embodiment the method includes the further step of selecting a more primitive precursor or a less differentiated cell, e.g., pluripotent stem cell (or a population thereof) made by the method which has lost a vector which encodes the heterologous nucleic acid.

In one embodiment, the somatic cell is as described above.

In an embodiment, the somatic cell is obtained from a first individual and the more primitive precursor or a less differentiated cell, e.g., pluripotent stem cell (or a population thereof) (or a tissue derived therefrom) is administered to the same first individual, or to a second individual, e.g., an individual related to said first individual. The second individual can be an individual who carries a different allele for a selected gene than does the first individual. E.g., the first individual can have an allele which does not cause a disease state or unwanted condition and the second individual has the allele which causes the disease state or unwanted condition.

In one embodiment, the number of stem cells produced, e.g., in the presence of UTF1 or a p53 inactivation factor, or a combination thereof, is 5-, 10-, 15-, 20-, 25-, 30-, 35-, 40-, 50-, 100-, 120-, 130-, 140-, 150-, 200-, 250-, 500-, 750- or 1000-fold greater than the number of stem cells produced without UTF1 and/or a p53 inactivation factor.

In another aspect, the disclosure features a population of cells, e.g., pluripotent stem cell or a population of pluripotent stem cells, produced by a method described herein.

In another aspect, the invention features a reaction mixture including a somatic cell and a sufficient amount of Oct4, Klf4, Sox2, c-Myc, a MEK/ERK inhibitor, BIX01294, BAYK 8644, Kenpaullone, a GSK3 inhibitor, an HDAC inhibitor, a DNA methyl transferase inhibitor, UTF1 and a p53 inactivation factor, or a combination thereof, to convert the somatic cell to a more primitive precursor or a less differentiated cell, e.g., pluripotent stem cell (or a population thereof). In one embodiment, the somatic cell is treated with one or more transcription factors, for example, a transcription factor selected from Oct4, Klf4, Sox2 and c-Myc. In some embodiments, the somatic cell is treated with one, two, three or four transcription factors (e.g., the somatic cell is treated with Oct4□the somatic cell is treated with Oct4 and Sox2, the somatic cell is treated with Oct4, Sox2, and Klf4 or the somatic cell is treated with Oct4, Sox2, Klf4, and c-Myc). In some embodiments, the somatic cell is not treated with c-Myc and/or Klf4.

In another aspect, the disclosure features a composition, e.g., a pharmaceutical composition, comprising a cell, e.g., a pluripotent stem cell or a population of pluripotent stem cells, produced by a method described herein.

The methods and pluripotent stem cells described herein are useful for treating a wide variety of conditions, including hematopoietic conditions (e.g., sickle cell anemia, leukemias, immune deficiencies), cardiac disorders (e.g., myocardial infarcts, and myopathies) and disorders such as liver disease, diabetes, thyroid abnormalities, neurodegenerative/neurological disorders (e.g., Parkinson's, Alzheimer's, stroke injuries, spinal chord injuries), circulatory disorders, respiratory disorders, wound healing and/or repair, bone repair, and enzyme abnormalities.

In one embodiment, the disclosure features a method of treating a disorder described herein, wherein the method includes: administering a pluripotent stem cell or a population of pluripotent stem cells produced by a method described herein to a subject, e.g., a subject that suffers from a disorder described herein.

In one embodiment of the methods described herein, the somatic cell contains one or more genetic defect, and, e.g., the pluripotent stem cell produced by a method described herein includes the genetic defect or defects. In some embodiments, the genetic defect is corrected (e.g., by homologous recombination) in the pluripotent stem cell, e.g., to provide a corrected pluripotent stem cell. Such cells can be administered by known methods such as the methods described e.g., in U.S. Publication No: 20030228293, the contents of which is incorporated herein by reference. The genetic defect corrected can be, for example, a genetic defect that causes an immune system disorder; a genetic defect that causes a neurological disorder; a genetic defect that causes a cardiac disorder; a genetic defect that causes a circulatory disorder; a genetic defect that causes a metabolic disorder such as diabetes; or a genetic defect that causes a respiratory disorder.

In some embodiments of the methods described herein, the pluripotent stem cell or population of pluripotent stem cells are differentiated in vitro into tissue or cell types useful in treating the condition or disorder described herein. In one embodiment, the pluripotent stem cell or tissues or cell types derived from the pluripotent stem cells are introduced into the subject from which the somatic cell was obtained. In one embodiment, the somatic cell is obtained from a subject having one or more genetic defects and the corrected pluripotent stem cell or a tissue of cell type derived from the corrected pluripotent stem cell is reintroduced to the subject. Differentiation can be effected by known methods. In one embodiment, the pluripotent stem cells are used to produce hematopoietic stem cells (HSC) which are, e.g., useful for transplantation and restoration of immune function in immune deficient recipients.

The methods described herein can further include maintaining the pluripotent stem cells under conditions which result in their differentiation into a desired cell type(s) (e.g., into repaired neurons, cardiac myocytes, blood cell type, bone cell (e.g., osteoblast) or pancreatic cells).

In one aspect, the invention includes a method for manufacturing a medicament for treating a disorder described herein, comprising providing a cell (e.g., an iPSC) produced by the method described herein. The medicament can include other features described herein.

Kits for practicing the methods disclosed herein and for making cells disclosed herein (e.g., iPS cells) are included.

In one aspect, a kit will contain a somatic cell, the components described herein (e.g., a p53 inactivation factor etc.) used to converting the somatic cell to an iPS cell and instructions for converting a somatic cell to an iPS cell using the method described herein.

In one embodiment, the somatic cell is directed to an iPS cell. In one embodiment, the somatic cell can be used as a control.

In another aspect, the invention features a kit comprising an iPS cell made by a method described herein and one or more component(s) for expanding (e.g., multiplying or proliferating) the iPS cell. The component(s) for expanding (e.g., multiplying or proliferating) the iPS cell are known in the art.

In another aspect, a kit contains an iPS cell, for example, made by a method described herein and instructions for directing an iPS cell to a differentiated cell.

The kit will typically be provided with its various elements included in one package, e.g., a fiber-based, e.g., a cardboard, or polymeric, e.g., a styrofoam box. The enclosure can be configured so as to maintain a temperature differential between the interior and the exterior, e.g., it can provide insulating properties to keep the reagents at a preselected temperature for a preselected time.

The kit can include one or more containers for the composition containing a component(s) described herein.

In one aspect, the invention features a method for reprogramming a somatic cell to form a less differentiated cell comprising treating factor A and factor B into a somatic cell, wherein factor A is selected from one or more of the group consisting of Oct4, Klf4, Sox2, c-Myc, a MEK/ERK inhibitor, BIX01294, BAYK 8644, Kenpaullone, a GSK3 inhibitor, an HDAC inhibitor and a DNA methyl transferase inhibitor, and factor B is selected from one or two of the group consisting of UTF1 and a p53 inactivation factor, thereby producing a cell that is less differentiated than the somatic cell (e.g., an ES-like cell).

In yet another aspect, the invention features a reprogrammed somatic cell produced by the method described herein, in which expression of a plurality of genes that are up-regulated or down-regulated in ES cells is up- or down-regulated in the reprogrammed somatic cell, wherein these genes are not up- or down-regulated in the somatic cell prior to reprogramming.

In some embodiments, expression of the genes Rex3 and Zfp7 is up-regulated, and expression of the genes Aspn and Meox2 are down-regulated compared to the expression of these genes in the somatic cell prior to reprogramming.

In one aspect, the invention features a reprogrammed somatic cell in which expression of a plurality of genes that are specifically expressed in ES cells are up-regulated in the reprogrammed somatic cell, wherein these genes are not up-regulated in the somatic cell prior to reprogramming.

In another aspect, the invention features a reprogrammed somatic cell produced by the method described herein, in which expression of a plurality of genes that are specifically expressed in the somatic cells prior to reprogramming, but are not expressed in ES cells, are down-regulated in the reprogrammed somatic cell, wherein these genes are not down-regulated in the somatic cell prior to reprogramming.

In yet another aspect, the invention features a reaction mixture comprising a somatic cell and (i) UTF1, (ii) a p53 inactivation factor, or (iii) a mixture thereof.

### BRIEF DESCRIPTION OF THE DRAWINGS

FIG. 1. Increased efficiency of human iPS cell generation by p53 siRNA (P) and UTF1 (U). (A): Increased efficiency of the AP-positive colony generation by UTF1 and p53 siRNA in a representative experiment. 5×10⁴ cells were plated onto a 10-cm dish. Shown are numbers of total colonies and AP-highly positive colonies (in parenthesis) counted on day 18 post-transduction. “4” indicates the reported four reprogramming factors OCT4 (O), SOX2 (S), KLF4 (K) and c-MYC (M). (B): Typical colonies of human iPS cells (top panel) and incompletely reprogrammed highly AP-positive cells (bottom panel). (Ba and Be) phase image, (Bb and Bf) high magnification, (Bc and Bg) AP staining, (Bd and Bh) GFP fluorescence. Bars, 100 μm. (C-E): Total colony numbers (left panels), highly AP-positive colony numbers (middle panels) and iPS colony numbers (right panels). The colony number shown are those induced from 10⁵ cells plated on the feeder cells by different combinations of factors. The results of three independent experiments are shown with different colors. (C) Enhanced efficiency in the presence of the reported four reprogramming factors. Numbers were counted on day 25 post-transduction; (D) The six factors (4PU) combination minus the reported four reprogramming factors, respectively. (Da and Db) Numbers were counted on day 25 post-transduction. (Dc) Numbers were counted on day 35 post-transduction. (E) p53 siRNA and UTF1 enhanced efficiency in the combinations without c-MYC. Numbers were counted on day 35 post-transduction.

FIG. 2. Identification of gene expression and differentiation potential in human iPS cells. (A) Semi-quantitative RT-PCR analysis of hES cell markers in iPS cells and pre-iPS cells. (B) Immunocytochemical staining for hES cell markers NANOG, SSEA-1, SSEA-4, TRA-1-60 and TRA-1-81 (red) in hAFF-4U-M-iPS-3. Nuclei are stained with DAPI (blue). Bars, 100 μm. (C) DNA microarray and hierarchical clustering of global gene expression of human iPS cells, pre-iPS cells, hES cells (H1, H7) and somatic cells (hAFF, hFSF). (D) Immunocytochemical staining for three germ layer markers in the differentiated human iPS cells. Ectoderm: PAX6 and NEUROFILAMENT (hAFF-4PU-iPS-13); GFAP and β-III-TUBULIN (hAFF-4U-M-iPS-3). Mesoderm: α-SMA (hAFF-4PU-iPS-13); BRACHYURY and VIMENTIN (hAFF-4U-M-iPS-3). Endoderm: SOX17 and FOXA2 (hAFF-4PU-iPS-13); CK18, ALB and AFP (hAFF-4U-M-iPS-3). Bars, 25 μm (ALBUMIN, BRACHYURY), 100 μm (others). (E) Teratoma formation of iPS cells. Nine-week teratomas were recovered from the SCID beige mouse. Cell types of the three germ layers were found. (Ea-Eh) Teratoma derived from hAFF-4PU-iPS-13; (Ei-Ep) Teratoma derived from hAFF-4U-M-iPS-3. Ectoderm: nerve fiber, (Ea) (NEUROFILAMENT positive), (Ej and Em) (bottom-left); neural tube-like epithelium, (Ec) (β-TUBULIN III positive), (Ek and Em) (up-right); neuron-like cells, (Eb) (β-TUBULIN III positive); pigmented retinal epithelium, (Ed and Ei). Mesoderm: smooth muscle, (Ee) (α-SMA positive), (Ef) (α-SMA positive), (El, Em) (middle); cartilage, (Eg) (VIMENTIN positive), (Eo); bone, (Ep). Endoderm: gut-like epithelium, (Eh) (CK19 positive), (En). Bars, 50 μm.

FIG. 3. Vector maps and transduction efficiency of lentiviral vectors used for reprogramming experiments. (A) Vector map of pLL3.7-ΔU6. The genes used in our work were constructed downstream of CMV promoter. (B) Vector map of pLL3.7, with the cassette of CMV-GFP. (C) Image of GFP and immunohistochemical staining of OCT4, SOX2, c-MYC, KLF4, UTF1 in cells at day 6 post-transduction to show the lentiviral transduction efficiency when six factors were co-introduced. Bars, 4 μm. (D) Knockdown of p53 expression by p53 siRNA in hAFF. A nonsense siRNA was used as a control.

FIG. 4. Colonies emerged after transduction of reprogramming factors. (A) Three groups of cell colonies analyzed by AP staining. Shown are fibroblasts (a), AP-negative granulated colonies (b), AP-low/noncompact colonies (c) and highly AP-positive colonies (d). (B) Typical image of hES like cells (white arrows in a, c) emerged since day 18 post-transduction. Fluorescence microscopy image (b, d) showed they were GFP negative. (C) Images of human iPS cells derived from hAFF (a), BJ (b) and hEF (c). Bars, 4 μm.

FIG. 5. Similar reprogramming efficiency estimated by using GFP-/ES-like criterion and the expression of NANOG or TRA-1-81 as the criteria. Figures at the right panel were merged from Figures of GFP, Nanog (or TRA1-81) and DAPI. (A) Immunostaining for NANOG in GFP-/ES-like colonies. Bars, 100 μm. (B) Immunostaining for TRA-1-81 in GFP-/ES-like colonies. Bars, 100 μm. (C) Number of iPS cell colonies estimated by using GFP-/ES-like criterion and the expression of NANOG as the criteria. (D) Number of iPS cell colonies estimated by using GFP-/ES-like and the expression of TRA-1-81 as the criteria.

FIG. 6. Silenced exogenous gene expression in human iPS cells. (A) Silenced exogenous gene expression in iPS cells derived from different fibroblasts. (B) Silenced exogenous gene expression in iPS cells derived from hAFF without c-MYC. (C) Attenuated p53 siRNA expression in iPS cells. Exogenous GFP transgene (driven by CMV promoter in the same p53 shRNA expressing construct) is efficient silenced. H1, H9, hAFF, iPS cells induced without p53 siRNA introduction were used as controls.

FIG. 7. Methylation profiles of promoter regions in human iPS cells. hFSF and the derived two iPS cell lines were analyzed for the promoters of OCT4 and NANOG, with H1 as the control for a demethylated state.

FIG. 8. Karyotype analysis of human iPS cells. hFSF-4PU-iPS-1 at passage 15 was used for karyotype analysis.

FIG. 9. Similar differentiation potential of iPS cells and hES cells. (A) Typical image of embryoid bodies derived from iPS cells at Day 5. Shown is embryoid bodies formed by hFSF-4PU-iPS-1. Bar, 200 μm. (B) Spontaneous differentiation of human iPS cell line hFSF-4PU-iPS-1 through EBs. Genes of three germ layers were detected by RT-PCR analysis. (C) Directed differentiation of iPS cell line hFSF-4PU-iPS-1 to mesoderm. Undifferentiated iPS cells were shown as negative controls. (D) Trophoblast lineage differentiation of iPS cell line hFSF-4PU-iPS-1 detected by hCG ELISA. The supernatant of differentiated iPS cell culture medium were tested on day 0, day 4 and day 9 post-inducement. Supernatant of cells maintained in MEF-conditioned medium (CM) were also tested to detect the spontaneous differentiation. hES cell line H1 was used as a control.

FIG. 10. The colony number shown are those induced from 10⁴ cells plated on the feeder cells by different combinations of factors. IPS colony numbers were counted on day 25 post-transduction. (O: OCT4; S:SOX2; K: KLF4; G: GFP; P: P53siRNA; U: UTF1). (A) In presence of VPA, iPS cell generation efficiency could be enhanced by UTF1 or P53 siRNA, or their combination. (B) p35 siRNA enhances reprogramming efficiency in presence of two HDAC inhibitor combination. VS: VPA+SBHA; VB: VPA+Butyrate. The working concentrations of VPA, SBHA and Butyrate in this study were 0.5 mM, 2 μM, 0.1 mM, respectively.

FIG. 11. The colony number shown are those induced from 1×10⁴ pOct4-GFP MEF cells when oct4(O), sox2(S), klf4(K) were introduced. GFP positive/ES-like colony numbers (considered as iPS colony number) were counted on day 25 post-transduction. iPS colony number was shown about 3 times more when KU55933 was added into the programming process than that without KU55933 (control).

FIG. 12. Vector maps of pLL3.7 (A) and pLL3.7-ΔU6 (B).

FIG. 13. Vector maps of (A) pMDLg/pRRE, (B) RSV/Rev and (C) VSV-G, which are used for lentivirus packaging. pVSV-G expresses the G glycoprotein of the vesicular stomatis virus under the control the CMV promoter. VSV-G is used to pseudo type MMLV-based retroviral vectors by mediating entry. VSV-G interacts with phospholipid target membrane and rosters the fusion of Viral and cellular membranes require a cell surface receptor and can serve as a surrogate viral protein. Includes IVS, a synthetic intron known to enhance the stability of the mRNA, the Col1 origin of replication and E. coli Amp^{r} gene for propagation and antibiotic selection in bacteria. As part of the Pantropic Retroviral Expression System, pVSV-G is cotransfected with a retroviral expression vector into the GP2-293 Packaging Cell Line to infectious, replication-incompetent retrovirus. The genes encoding the viral gag and proteins are stably integrated into GP2-293. Because the VSV-G envelope protein causes toxicity by fusing cellular membranes, it must be expressed transiently from pVSV-G during packaging. Although the resulting virus can infect target cell lines and transmit a target gene, it cannot replicate because target cell lines lack the viral structural and polymerase/integrase genes. The separate introduction and integration of the viral genes into the packaging cell line and the use of minimal viral sequences in the vector minimize the chance of producing replication-competent virus due to recombination events.

### DETAILED DESCRIPTION OF THE PREFERRED EMBODIMENT

As described herein, UTF1 and a p53 inactivation factor can be employed to efficiently generate induced pluripotent stem (iPS) cells from skin fibroblasts or other cell types.

iPS cells can be created by over-expression of one or more genes, for example one or more of the following four genes: Oct4, Sox2, c-Myc and Klf4 through retroviral infection, but with low efficiencies. All of these four genes are known to be or considered to be DNA binding proteins, transcription factors. Notably, the oncogene c-Myc used in this approach causes tumor formation in cells derived from the iPS cells. Although iPS cells can be generated with only Oct4, Sox2 and Klf4, the efficiency is even lower; fewer than iPS colonies from out of 100,000 cells. These issues pose significant barriers for creation of iPS cells for therapeutic applications. One obvious concerns is the use of retroviruses, which integrate into chromosomal DNA and can cause ancillary problems (mutations). Beyond the use of retroviral vectors and the insertion mutations they cause, the methods involves adding new reprogramming factors (eg. new transcriptional factors or new chemical compounds) to the cell.

As described herein, additional reprogramming factors such as Utf1 and/or a p53 inactivation factor can improve the efficiency of iPS cell induction up to more than 100 fold. For example, infection with Utf1 and p53 siRNA, induced 0.1-0.2% iPS cells in human fibroblasts infected with the four factors (Oct4, Sox2, c-Myc and Klf4), a more than 100 fold improvement over the non-treated control (−0.001%). Infection with p53 siRNA and Utf1 induced 0.1-0.2% iPS cells in human fibroblasts infected with the three factors (Oct4, Sox2 and Klf4, but not c-Myc).

As described herein, infection of a p53 inactivation factor (such as p53 siRNA) and Utf1 enables reprogramming of human cells by only three other transcription factors, Oct4 and Sox2 and Klf4, without the need for the oncogenes c-Myc. For example, iPS colonies were identified about 1 month post-infection in human fibroblasts infected by Oct4, Sox2 and Klf4 together with p53 siRNA and Utf1. On average, about 71-223 iPS cells were successfully obtained out of every 100,000 human adult fibroblast cells after infection.

Moreover, infection of p53 siRNA or Utf1 enables reprogramming of human cells by only three other transcription factors, Oct4 and Sox2 and Klf4, without the need for the oncogenes c-Myc. For example, iPS colonies were identified about 1 month post-infection in human fibroblasts infected by Oct4, Sox2 and Klf4 together with p53 siRNA or Utf1. On average, about 30 iPS cells were successfully obtained out of every 100,000 human adult fibroblast cells after infection.

The methods described herein improve the efficiency of creating iPS cells from skin (e.g., human skin cells) and are useful for making induced stem cells from other cell types with or without using the oncogenes c-Myc. For example, these additional factors may make it possible to create iPS cells from small numbers of cells (e.g., such as those obtained from hair follicle cells from patients, blood samples, adipose biopsy, etc), something that could otherwise be difficult or impossible due to the low efficiency of the current method. Thus, the addition of genes (e.g. p53 inactivated factor and/or Utf1 etc.) or small molecules compounds (e.g., chemicals described herein) can increase the probability of success when trying to make iPS cells from human skin biopsies (fibroblasts or other nucleated cells) and may be helpful in creating iPS cells from any other cell types.

### Stem Cells

Stem cells are cells that retain the ability to renew themselves through mitotic cell division and can differentiate into a diverse range of specialized cell types. The two broad types of mammalian stem cells are: embryonic stem cells that are found in blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

Stem cells can be used, e.g., in bone marrow transplants to treat leukemia. Stem cells can be used to treat diseases including cancer, Parkinson's disease, muscle damage, burns, heart disease, diabetes, osteoarthritis, rheumatoid arthritis, hematopoietic conditions (e.g., sickle cell anemia, leukemia, lymphoma, inherited blood disorders), immune deficiencies), cardiac disorders (e.g., myocardial infarcts, and myopathies) and disorders such as liver disease, diabetes, thyroid abnormalities; neurodegenerative/neurological disorders (e.g., Parkinson's Disease, Alzheimer's Disease, stroke injuries, spinal chord injuries), Crohn's Disease, circulatory disorders, respiratory disorders, wound healing and/or repair, bone repair, and enzyme abnormalities.

### Cell Types for Use in the Preparation of Stem Cells

The methods described herein can be used, e.g., to reprogram somatic cells to a pluripotent state. Such somatic cells can be obtained, for example from a patient, to prepare patient-specific stem cells (e.g., patient-specific pluripotent stem cells). A variety of cells can be used, such as, hair follicle cells, a cell from a blood sample, a cell from adipose tissue, a stomach cell, a liver cell, or a cell from skin (e.g., fibroblast or other cell type, e.g., keratinocyte, melanocyte, Langerhans cell, or Merkel cell) and so on.

Somatic cells are any cells forming the body of an organism, as opposed to germline cells. In mammals, germline cells (also known as gametes) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell. For example, internal organs, skin, bones, blood, and connective tissue are all made up of somatic cells.

Additional cell types include: a fibroblast (e.g., aprimary fibroblast), a muscle cell (e.g., a myocyte), a cumulus cell, a neural cell, a mammary cell, a hepatocyte and a pancreatic islet cell. In some embodiments, the somatic cell is a primary cell line or is the progeny of a primary or secondary cell line. In one embodiment, the somatic cell is obtained from a sample, e.g., a hair follicle, a blood sample, a biopsy (e.g., a skin biopsy or an adipose biopsy), a swab sample (e.g., an oral swab sample).

### p53 Inactivation Factors

The p53 tumour suppressor protein is a gatekeeper of cellular fate in multicellular organisms. p53 is activated in response to genotoxic stress and initiates cell cycle arrest, senescence and/or apoptosis via pathways involving transactivation of p53 target genes. p53-controlled transactivation of target genes is an essential feature of each stress response pathway, although some effects of p53 may be independent of transcription. As a transcription factor that both activates and represses a broad range of target genes, p53 demands an exquisitely complicated network to control and fine-tune responses to the various stress signals encountered by cells. p53 contributed to spontaneous and DNA damage-induced apoptosis of hESCs through a transcription-independent mitochondrial pathway.

Examples of p53 inactivation factors include Pifithrin-α, Pifithrin-α Cyclic-, Pifithrin-α p-Nitro-, Pifithrin-α p-Nitro-Cyclic-, ATM/ATR Kinase Inhibitor, Caspase Inhibitor VI, ATM Kinase Inhibitor, TCDD (2,3,7,8-tetrachlorodibenzo-p-dioxin), CHL (Chlorophyllin), RTK1 (plumbagin), Dicoumarol, ATO (Arsenic trioxide), cyclosporin A, 3,4-dihydro-5-[4-(1-piperidinyl)butoxy]-1(2H)-isoquinolinone (DPQ), Bax-Inhibiting Peptide (V5), Bax inhibitor peptide P5, Chk2 Inhibitor, Chk2 Inhibitor II, Debromohymenialdisine, Stylotella aurantium, hydrobromide, CGK 733. Other inhibitors include, for example, dominant negative forms of the p53 (e.g., catalytically inactive forms) siRNA inhibitors of p53, and antibodies that specifically bind to p53. Inhibitors are available, e.g., from Tocris, Calbiochem and Sigma Aldrich.

### UTF1

The UTF1 (undifferentiated embryonic cell transcription factor 1) gene, is specifically expressed in the inner cell mass and primitive ectoderm and is down-regulated at early primitive streak stages. Expression is maintained in the primordial germ cells in developing embryos and in the gonads in adult animals. Promoter analysis indicated that the murine UTF1 gene is transcriptionally regulated by Oct4 and Sox2. The UTF1 protein was shown to repress transcription, to activate reporter genes in an ATF2-dependent manner, and to interact with the basal transcription factor TFIID. A recent study suggested a role for UTF1 in the proliferation rate and teratoma-forming capacity of ES cells. Human UTF1 is a tightly DNA-associated protein with transcriptional repressor activity, as a pluripotency-associated chromatin component with core histone-like characteristics.

### Histone Deacetylase Inhibitors

Histone deacetylases (HDAC) are a class of enzymes that remove acetyl groups from an [epsilon]-N-acetyl lysine amino acid on a histone. Exemplary HDACs include those Class I HDAC: HDAC1, HDAC2, HDAC3, HDAC8; and Class II HDACs: HDAC4, HDAC5, HDAC6, HDAC7A, HDAC9, HDAC1O. Type I mammalian HDACs include: HDAC1, HDAC2, HDAC3, HDAC8, and HDACI1. Type II mammalian HDACs include: HDAC4, HDAC5, HDAC6, HDAC7, HDAC9, and HDAC1.

As used herein, the term HDAC inhibitor refers to a compound that inhibits a histone deacetylase Class I and/or Class II enzyme. In some embodiments, the compound selectively inhibits a Class I or Class II HDAC.

By “selective” is meant at least 20%, 50%, 75%, 2-fold, 3-fold, 4-fold, 5-fold, 6-fold, or 10-fold greater inhibition of an HDAC over another enzyme, for example a Class III or Class IV histone deacetylase. Thus, in some embodiments, the agent is selective for HDAC over a Class III histone deacetylase. In some embodiment the inhibitor is specific for a Class I or Class II and thus does not significantly inhibit HDACs of other classes.

A number of structural classes of negative regulators of HDACs (e.g., HDAC inhibitors) have been developed, for example, small molecular weight carboxylates (e.g., less than about 250 amu), hydroxamic acids, benzamides, epoxyketones, cyclic peptides, and hybrid molecules. (See, for example, Drummond DC, Noble CO, Kirpotin DB, Guo Z, Scott G K, et al. (2005) Clinical development of histone deacetylase inhibitors as anticancer agents. Annu Rev Pharmacol Toxicol 45: 495-528, (including specific examples therein) which is hereby incorporated by reference in its entirety). Non-limiting examples of negative regulators of type I/II HDACs include: Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VPA) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (i.e., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-C1-UCHA (i.e., 6-(3-chlorophenylureido)caproic hydroxamic acid), AOE (2-amino-8-oxo-9,10-epoxydecanoic acid), CHAP31 and CHAP 50. Other inhibitors include, for example, dominant negative forms of the HDACs (e.g., catalytically inactive forms) siRNA inhibitors of the HDACs, and antibodies that specifically bind to the HDACs. Inhibitors are available, e.g., from BIOMOL International, Fukasawa, Merck Biosciences, Novartis, Gloucester Pharmaceuticals, Aton Pharma, Titan Pharmaceuticals, Schering AG, Pharmion, MethylGene, and Sigma Aldrich. In some embodiments, VPA is a preferred histone deacetylase inhibitor.

### DNA Methyltransferase Inhibitors

DNA methylation is one of the most prevalent epigenetic modifications of DNA in mammalian genomes. It is achieved by DNA methyltransferases that catalyze the addition of a methyl group from S-adenosyl-L-methionine to the 5-carbon position of cytosine. Methylation at cytosine plays an important role in regulating transcription and chromatin structure. Three families of DNA methyltransferase genes have been identified in mammals. They include Dnmt1, Dnmt2 and Dnmt3. Dnmt1 is constitutively expressed in proliferating cells and its inactivation results in demethylation of genomic DNA and embryonic death. Dnmt2 is expressed at low levels in adult tissues. Its inactivation does not affect DNA methylation or maintenance of methylation. The Dnmt3 (Dnmt3a and Dnmt3b) is strongly expressed in embryonic stem cells, but is down-regulated in differentiating embryonic stem cells and in adult somatic cells.

Most mammalian transcription factors bind GC-rich DNA elements. Methylation of these elements abolishes binding. CpG methylation is shown to induce histone deacetylation, chromatin remodeling, and gene silencing through a transcription repressor complex. CpG islands are often located around the promoters of housekeeping genes and are not methylated. In contrast, the CG sequences in inactive genes are usually methylated to suppress their expression.

Examples of nucleoside DNA methyltransferase inhibitors include 5-deoxy-azacytidine (DAC), 5-azacytidine (5-aza-CR) (Vidaza), 5-aza-2′-deoxycytidine (5-aza-CdR; decitabine), 1-[beta]-D-arabinofuranosyl-5-azacytosine, dihydro-5-azacytidine, zebularine, Sinefungin (e.g., InSolution™ Sinefungin), 5-fluoro-2′-deoxycyticine (FdCyd). Examples of non-nucleoside DNA methyltransferse inhibitors (e.g., other than procaine) include: (−)-epigallocatechin-3-gallate (EGCG), RG108, hydralazine, procainamide, 1513-DMIa and 1513-DMIb which were isolated from the culture filtrate of Streptomyces sp. strain No. 1513, psammaplin, dominant negative forms of the DNA methyltransferases (e.g., catalytically inactive forms), oligonucleotides (e.g., including hairpin loops and specific antisense oligonucleotides (such as MG98)), siRNA inhibitors of the DNA methyltransferases, and antibodies that specifically bind to the DNA methyltransferases. Inhibitors are available, e.g., from Merck Biosciences.

### Kits

The factor UTF1 and a p53 inactivation factor described herein can be provided in a kit.

In one embodiment, the kit includes one or two of the group consisting of UTF1 and p53 inactivation factor.

In a preferable embodiment, the kit further includes factor A selected from one or more (e.g. two, three or four) of the group consisting of Oct4, Klf4, Sox2 and c-Myc. In a more preferable embodiment, the kit further includes the compounds reported to improve reprogramming efficiency, (e.g. CHIR 99021, an HDAC inhibitor(s) such as VPA and/or a DNA methyltransferase inhibitor(s)), and, optionally (c) informational material. The informational material can be descriptive, instructional, marketing or other material that relates to the methods described herein and/or the use of a compound(s) described herein for the methods described herein.

The informational material of the kits is not limited in its form. In one embodiment, the informational material can include information about production of the compound, molecular weight of the compound, concentration, date of expiration, batch or production site information, and so forth. In one embodiment, the informational material relates to methods for administering the compound.

In one embodiment, the informational material can include instructions to administer the factor A selected from one or more (e.g. two, three or four) of the group consisting of Oct4, Klf4, Sox2 and c-Myc, and factor B selected from one or two of the group consisting of UTF1 and a p53 inactivation factor. In one embodiment, the informational material can include instructions to administer a compound(s) (e.g., CHIR 99021, an HDAC inhibitor(s) such as VPA and/or a DNA methyltransferase inhibitor(s)) described herein in a suitable manner to perform the methods described herein, e.g., in a suitable dose, dosage form, or mode of administration (e.g., a dose, dosage form, or mode of administration described herein) (e.g., to a cell in vitro or a cell in vivo). In another embodiment, the informational material can include instructions to administer a compound(s) described herein to a suitable subject, e.g., a human, e.g., a human having or at risk for a disorder described herein or to a cell in vitro.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In addition to a compound(s) described herein, the composition of the kit can include other ingredients, such as a solvent or buffer, a stabilizer, a preservative, a flavoring agent (e.g., a bitter antagonist or a sweetener), a fragrance or other cosmetic ingredient, and/or an additional agent, e.g., for inducing pluripotent stem cells (e.g., in vitro) or for treating a condition or disorder described herein. Alternatively, the other ingredients can be included in the kit, but in different compositions or containers than a compound described herein. In such embodiments, the kit can include instructions for admixing a compound(s) described herein and the other ingredients, or for using a compound(s) described herein together with the other ingredients, e.g., instructions on combining the two agents prior to administration.

A compound(s) described herein can be provided in any form, e.g., liquid, dried or lyophilized form. It is preferred that a compound(s) described herein be substantially pure and/or sterile. When a compound(s) described herein is provided in a liquid solution, the liquid solution preferably is an aqueous solution, with a sterile aqueous solution being preferred. When a compound(s) described herein is provided as a dried form, reconstitution generally is by the addition of a suitable solvent. The solvent, e.g., sterile water or buffer, can optionally be provided in the kit.

The kit can include one or more containers for the composition containing a compound(s) described herein. In some embodiments, the kit contains separate containers (e.g., two separate containers for the two agents), dividers or compartments for the composition(s) and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

As used herein, a heterologous nucleic acid, is a nucleic acid other than a native endogenous sequence for that gene. E.g., an additional copy of a gene inserted into a chromosome, or a copy on a vector, e.g., a replicative on non replicative vector which has not integrated into the chromosome.

Other features and advantages of the instant invention will become more apparent from the following examples and claims. Embodiments of the invention can include any combination of features described herein. In no case does the term “embodiment” necessarily exclude one or more other features disclosed herein, e.g., in another embodiment. The contents of all references, patent applications and patents, cited throughout this application are hereby expressly incorporated as a whole by reference.

### EXAMPLES

### Methods

### 1. Cell Culture

BJ fibroblasts were purchased from ATCC (CRL-2522). hEF (human embryonic fibroblast) cells, hFSF (human fetal skin fibroblast) cells, and hAFF (human adult foreskin fibroblast) cells were purchased from the China-Japan Friendship Hospital. pOct4-GFP mice were from RIKEN BioResource Centre (Ohbo K. et. al. 2003). Above human fibroblasts, 293T cells available commercially and MEF (mouse embryonic fibroblast) cells isolated from mouse using the known method (Takahashi et. al., 2007) were cultured in Dulbecco's Modified Eagle Medium (DMEM, Hyclone) containing 10% fetal bovine serum (FBS, Gibco). Human embryonic stem (hES) cell lines H1, H7 and H9 were obtained from WiCell research institute (Madison, Wis.). hES cells purchased from WiCell and iPS cells prepared by the method as described herein were maintained on Mitomycin C-treated MEFs in hES Cell culture medium consisting of 80% DMEM/F12 (Invitrogen), 20% Knockout serum replacement (KSR) (Invitrogen), 1 mM L-glutamine, 1% non-essential amino acids, 0.1 mM beta-mercaptoethanol (all from Invitrogen) and 4 ng/ml basic fibroblast growth factor bFGF (P&A Biotech). Human iPS cells and hES cells were passaged by dispase II (Invitrogen). The medium was changed every day.

### 2. Plasmid Construction

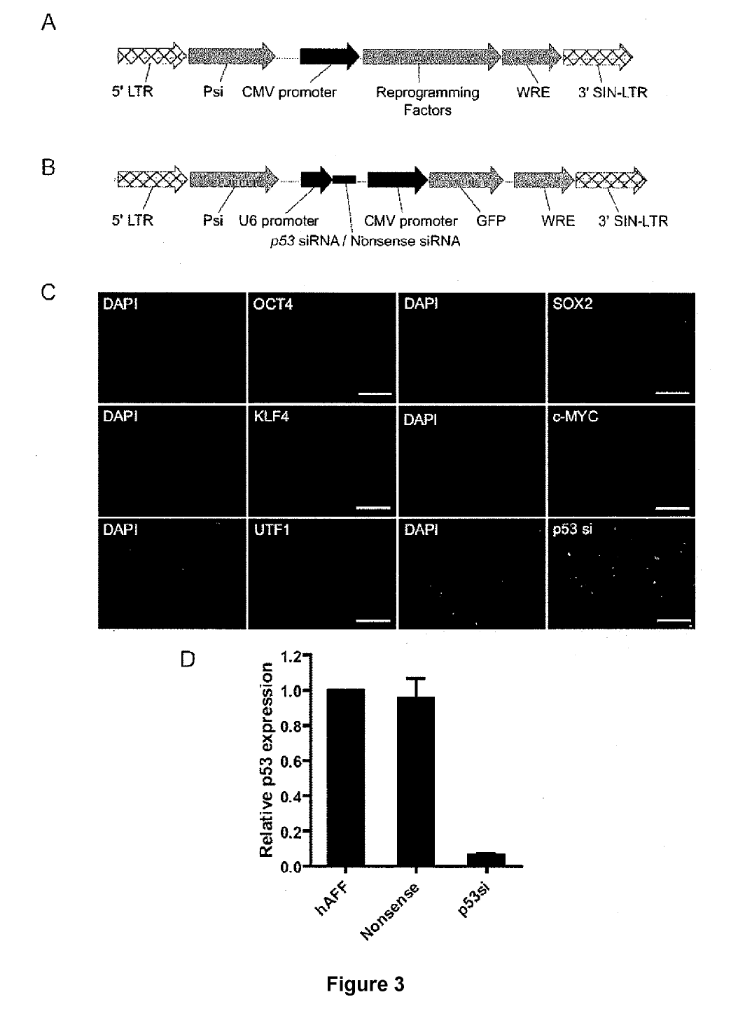

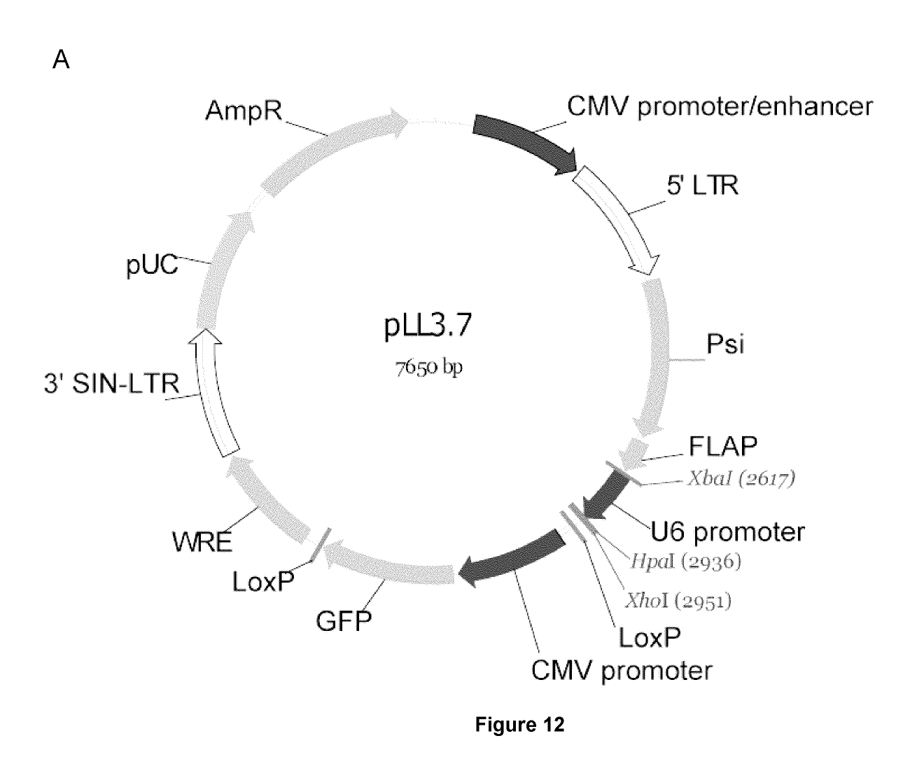

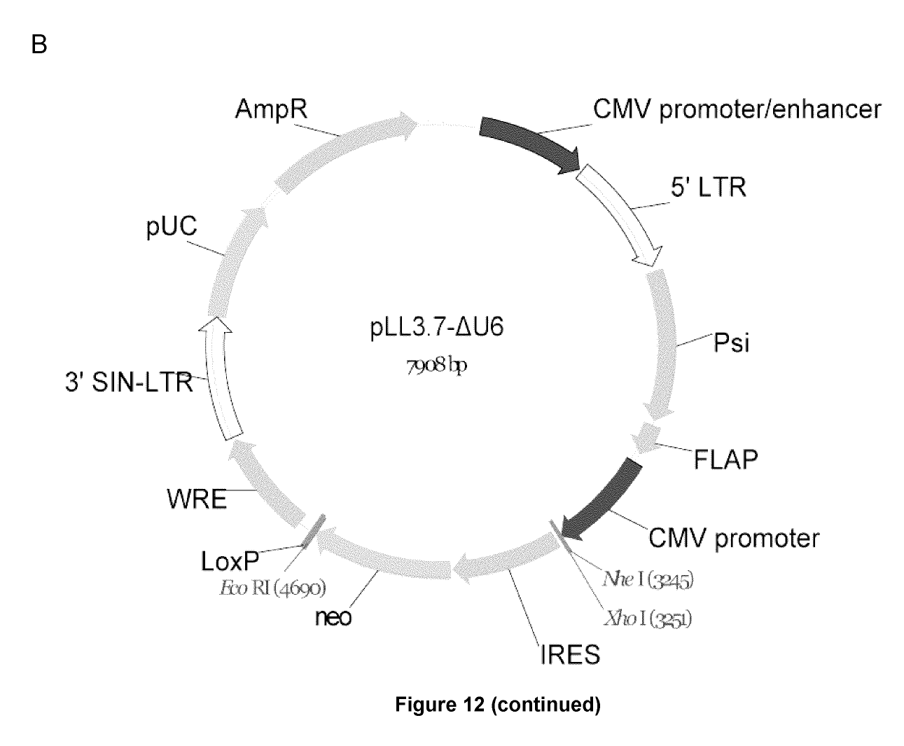

pLL3.7 vector (SEQ ID NO: 10) from Van Parijs L (FIGS. 3B, 12A) was modified by removing the U6 promoter and loxP between XbaI and NotI sites. An XhoI site was added upstream of the NheI site. The new lentiviral construct was named pLL3.7-ΔU6 (FIG. 12B). cDNA of the reported four human reprogramming factors (OCT4, SOX2, c-MYC, KLF4) (SEQ ID NO:1, 2, 3 and 4) and UTF1 (SEQ ID NO: 5) were amplified by RT-PCR and cloned into pEASY-Blunt vector (TransGen Biotech), confirmed by sequencing and then introduced into the XhoI/EcoRI or NheI/EcoRI sites of pLL3.7-ΔU6 (FIGS. 3A and 12B). Primers used are given in Table 3 (SEQ ID NO: 11-20). The oligonucleotides encoding p53 shRNA were 5′-TGACTCCAGTGGTAATCTACTTCAAGAGAGTAGATTACCACTGGAG TCTTTTTTC-3′ and 5′-TCGAGAAAAAAGACTCCAGTGGTAATCTACTCTCTT GAAG TAGATTACCACTGGAGTCA-3′ (SEQ ID NO: 6 and 7). The oligonucleotides encoding control shRNA were: 5′-TAATTCTCCGAACGTGTCACGTTCAAGAGACGTGACACGTTCGGAGAATTTTTTT TC-3′ and 5′-TCGAGAAAAAAAATTCTCCGAACGTGTCACGTCTCTTGAACGTGACACGTTCGG AGAATTA-3′ (SEQ ID NO: 8 and 9). The double strand DNA (dsDNA) for p53 shRNA obtained by annealing the sequences represented by SEQ ID NO: 6 and 7 and the control dsDNA obtained by annealing the sequences represented by SEQ ID NO: 8 and 9 were inserted into HpaI and XhoI site of the lentiviral vector pLL3.7, respectively.

### 3. Lentivirus Infection and Human iPS Cell Generation

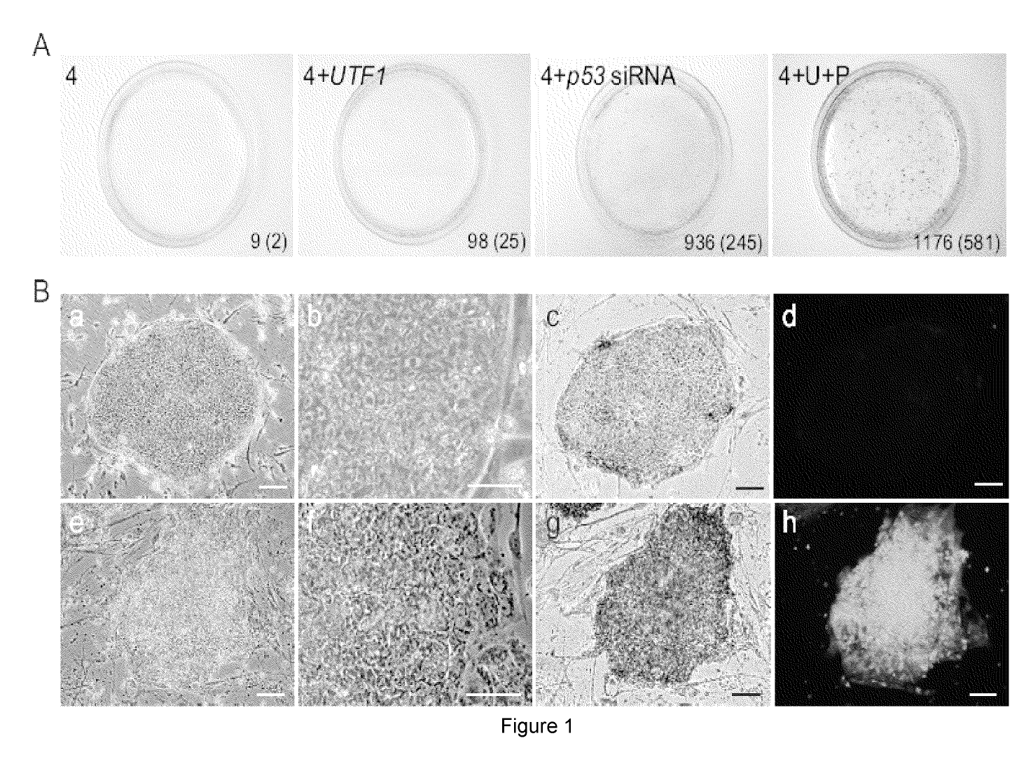

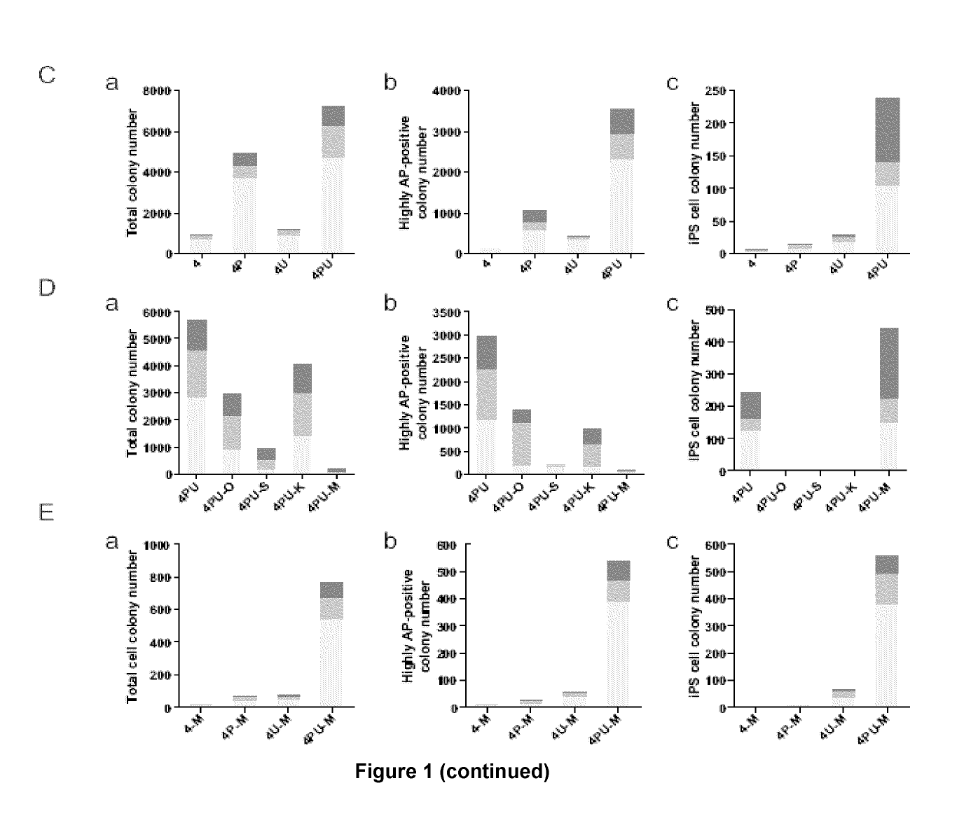

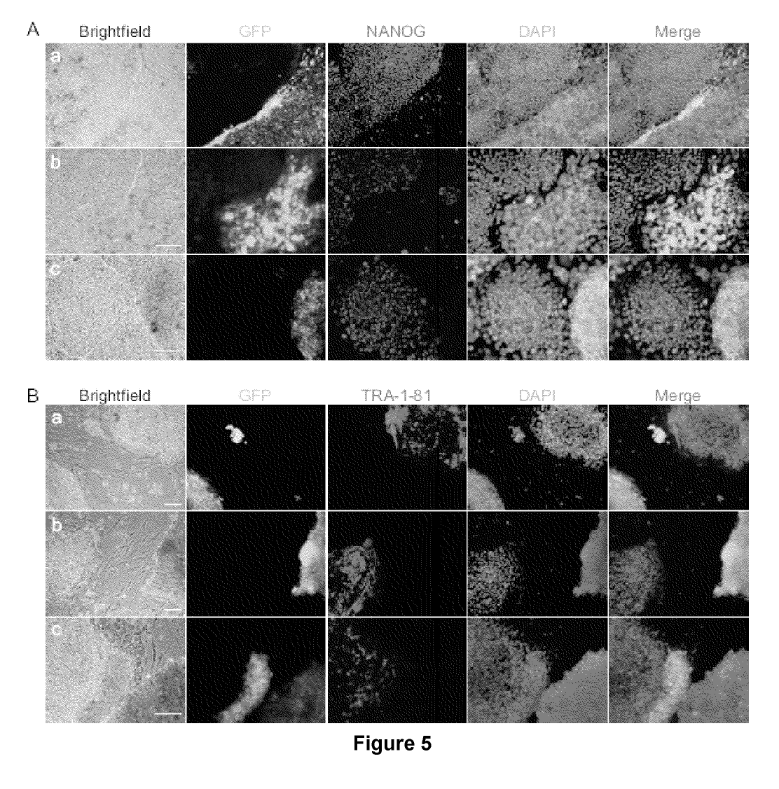

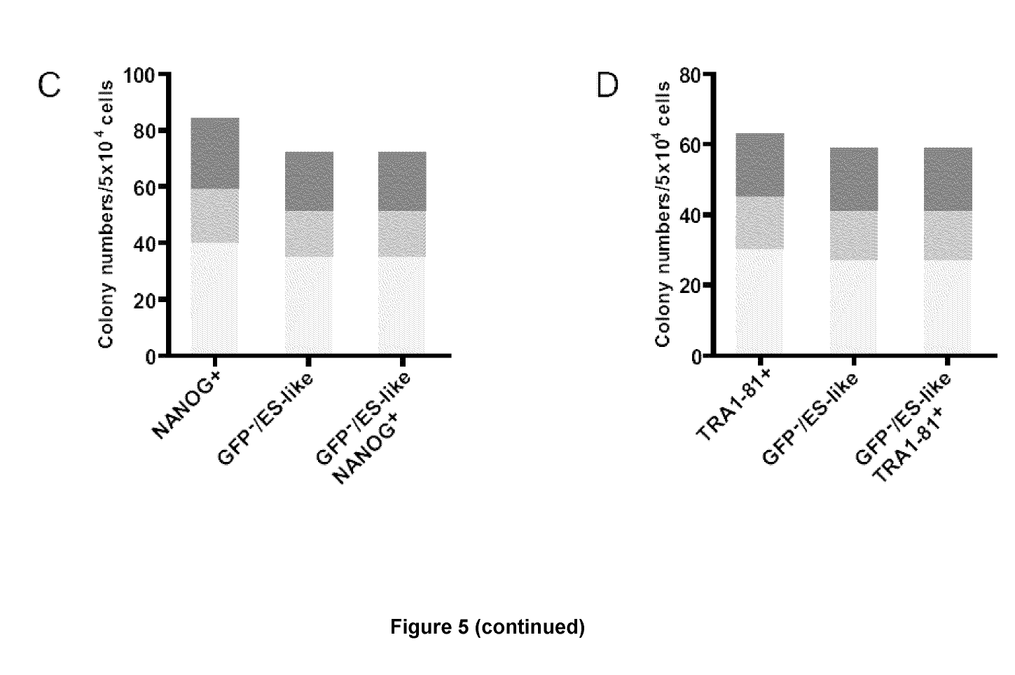

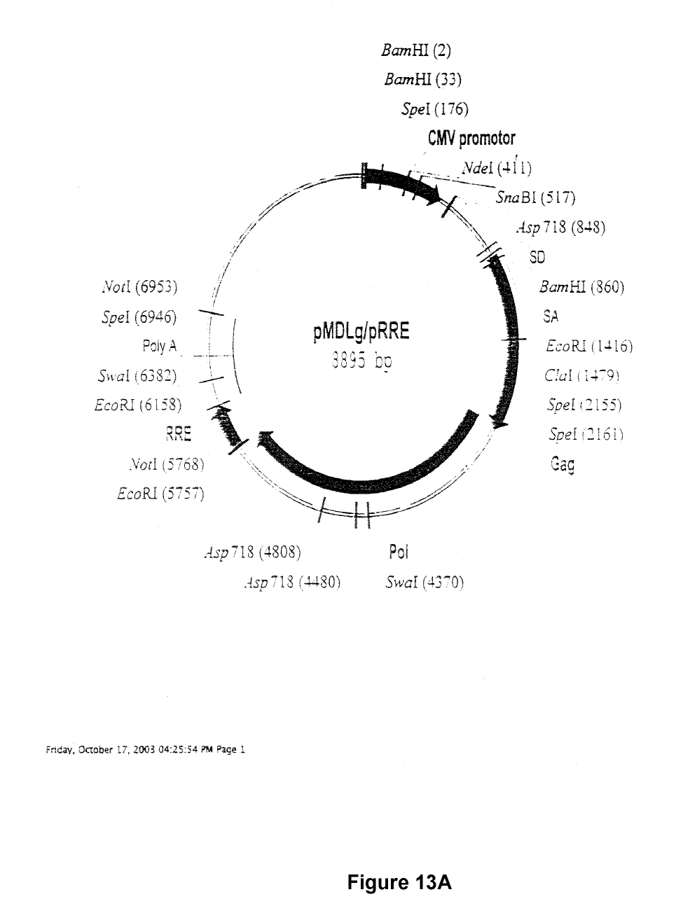

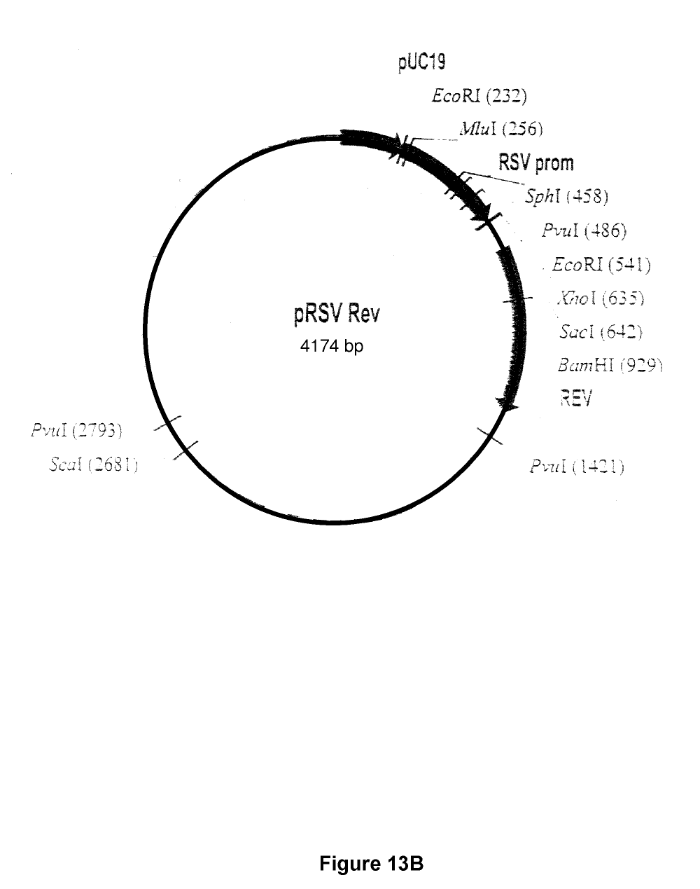

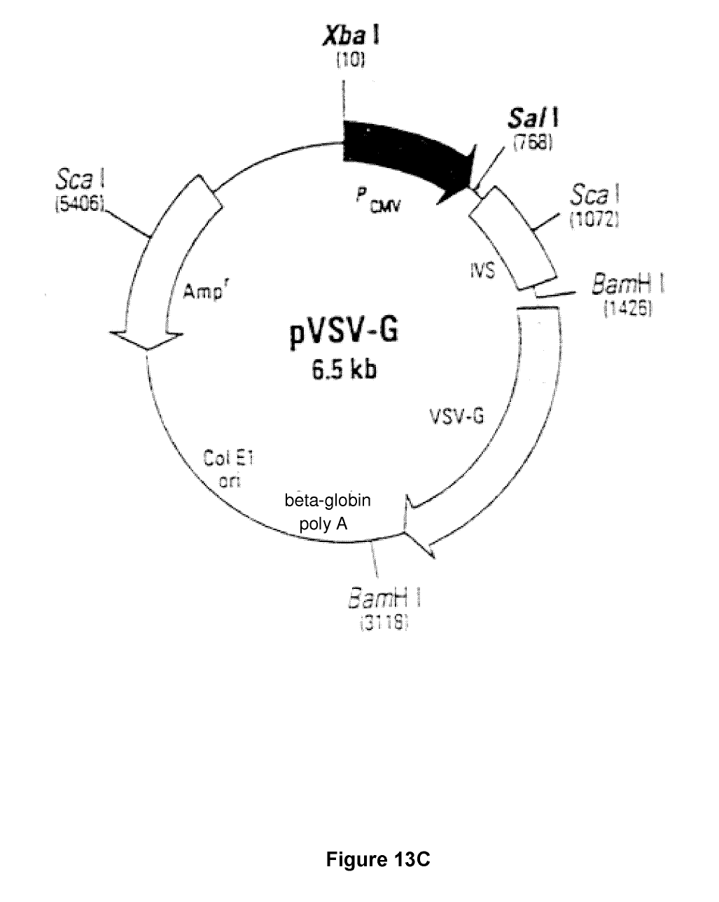

Lentiviral vector containing OCT4, SOX2, c-MYC, KLF4, UTF1, p53 siRNA and control p53 siRNA respectively (15 μg) together with pMDLg/pRRE, RSV/Rev and VSV-G (FIG. 13 A, B, C) (5 μg for each), were respectively transfected into 293T cells with the Ca₃(PO₄)₂-method in 10-cm dishes and incubated overnight. 12 hours later, the medium was changed and virus was collected after subsequent 36 hours cultivation. Viral supernatant was collected and filtered through 0.45 μm filters (Millipore) and then placed onto target fibroblasts supplemented with 10 ng/μl polybrene (Sigma-Aldrich). Typically, 1×10⁵ fibroblasts in a well of 6-well plate were infected with 250 μl viral supernatant of each gene. The viral supernatant was changed with fibroblast culture medium after 12 hours. The lentivirus transduction efficiency was very high, as indicated by the expression of EGFP and the introduced genes in the pLL3.7 vector (FIG. 3C), and the knockdown of p53 expression by p53 shRNA was efficient (FIG. 3D). 5-6 days later, the infected cells were collected, counted, and plated onto MEF feeders (typically 2×10⁴ to 1×10⁵ cells for a 10-cm dish). Medium was changed into hES cell culture medium 24 hours later. The medium was changed every other day until hES cell-like colonies appeared. The efficiency to generate iPS cells was calculated according to Takahashi's method, by which the number of cell colonies was counted and divided by the number of cells plated onto feeders (Takahashi et. al., 2007) (FIGS. 1C, 1D, 1E, 5C, and 5D). The hES cell-like colonies were picked at day 35 post-transduction and passaged in similar procedures to hES cells.

### 4. Alkaline Phosphatase (AP) Staining and Immunocytochemistry.

To detect alkaline phosphatase (AP) activity, the cells were washed with PBS three times and stained with BCIP/NBT (Promega) for 15˜20 min (FIG. 1A) or performed using Alkaline Phosphatase Detection Kit (Chemicon) (FIGS. 1B (c), 1B(g), and 4A). For immunocytochemistry, cells were fixed with 4% paraformaldehyde for 15 min, rinsed with PBS, and blocked with 2.5% donkey serum in 0.1% PBST (PBS+0.1% TritonX-100) for 60 minutes at room temperature. Primary antibodies included SSEA-1 (1:40, Chemicon), SSEA-4 (1:40, Chemicon), TRA-1-60 (1:50, Santa Cruz), TRA-1-81 (1:50, Santa Cruz), NANOG (1:40, R&D), PAX6 (1:200, Chemicon), beta-III TUBULIN (1:100, Santa Cruz), NEUROFILAMENT (1:100, Santa Cruz), GFAP (1:50, Zhongshan Biotech.), BRACHURY (1:50, R&D), alpha-SMA (1:200, Santa Cruz), VIMENTIN (working solution, Zhongshan Biotech.), CK18 (1:200, Invitrogen), CK19 (1:200, Invitrogen), ALB (1:500, DAKO), SOX17 (1:50, R&D), FOXA2 (1:100, Upstate) and AFP (1:100, Santa Cruz). Secondary antibodies were Rhodamine-labeled donkey anti-mouse IgG (1:100, Santa Cruz), Rhodamine-labeled donkey anti-rabbit IgG (1:100, Santa Cruz), Rhodamine-labeled donkey anti-goat IgG (1:100, Santa Cruz), Rhodamine-labeled donkey anti-goat IgM (1:100, Santa Cruz), and Rhodamine-labeled goat anti-mouse IgG3 (1:100, Santa Cruz). DAPI (Roch) was used for nuclear staining (FIGS. 1B, 1D, 3C, and 5).

### 5. Reverse Transcription PCR

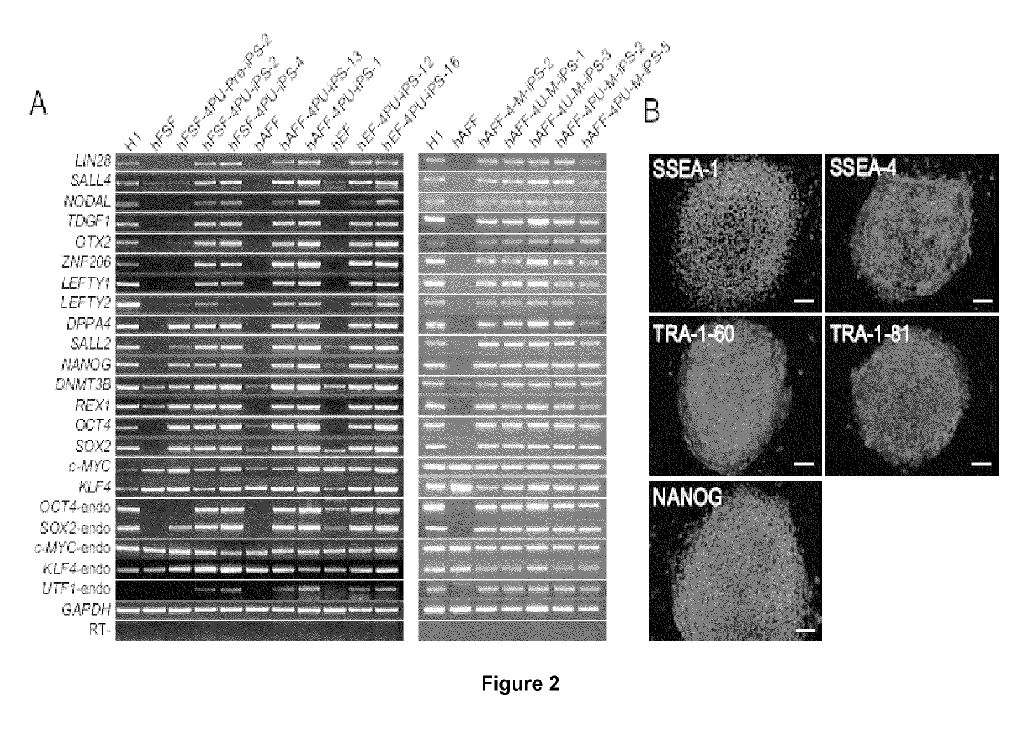

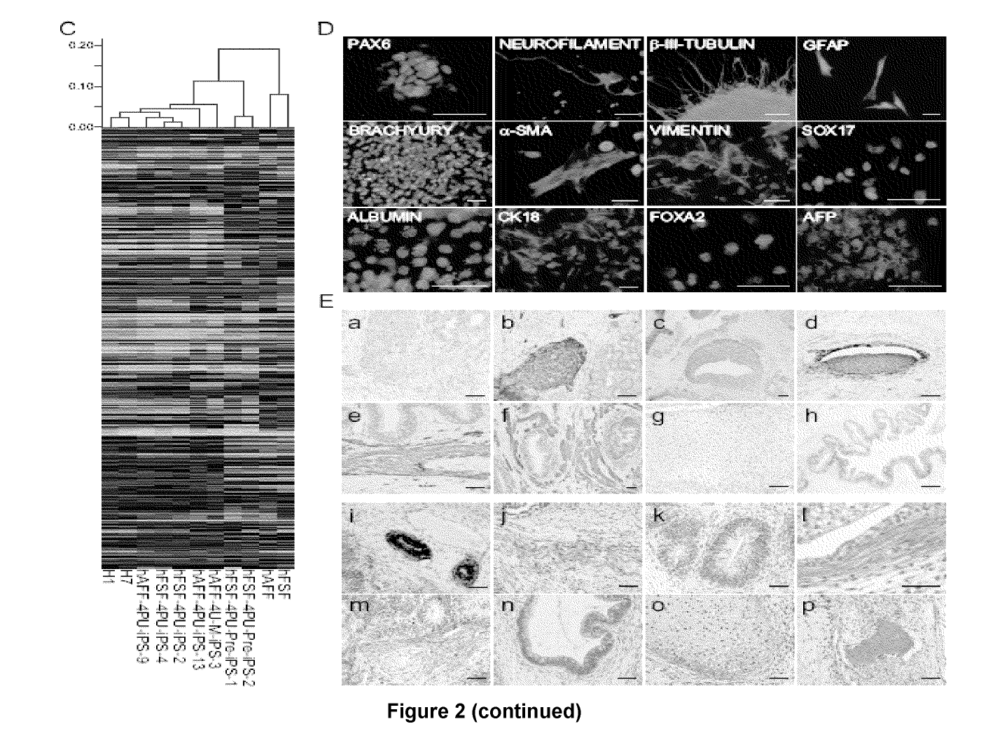

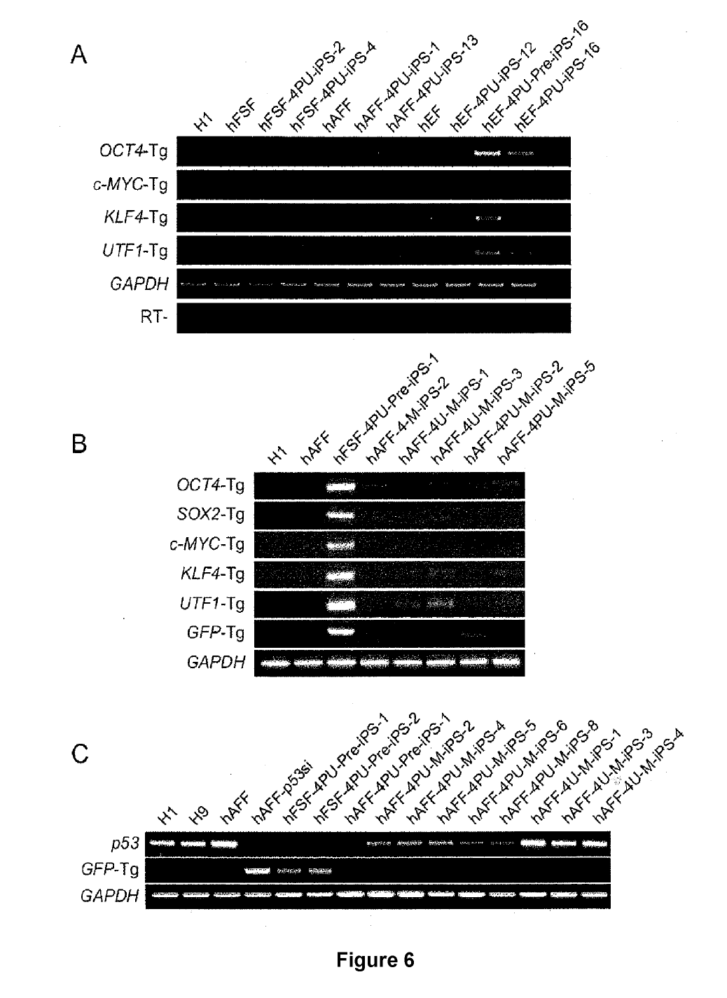

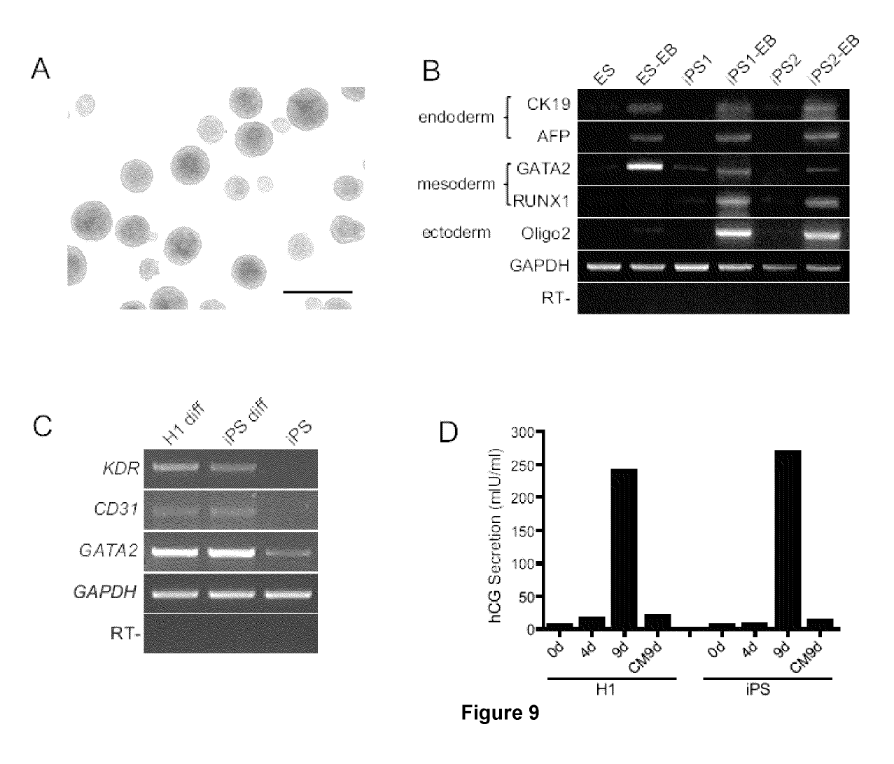

Total RNA was isolated from cells using TRIzol (Invitrogen) and reverse-transcribed using a reverse transcription system (Promega) according to the manufacturer's protocol. PCR amplification of different genes was performed using 2× EasyTaq SuperMix (TransGen Biotech). The primers (SEQ ID NO: 21-83) used are given in Table 4 (FIG. 2A, FIG. 6, FIGS. 9 (b) and (c)).

### 6. Differentiation In Vitro

For embryoid body (EB) formation, iPS cells were passaged with 1 mg/ml collagenase IV (Invitrogen) and cultured in an uncoated cell culture suspension dish (Corning) in the presence of Iscove's Modified Dulbecco's Medium (IMDM) supplemented with 15% fetal bovine serum (FBS), 1 mM L-glutamine, 1% non-essential amino acids, 1% ITS, and 0.1 mM b-mercaptoethanol (all from Invitrogen). Cyst embryoid bodies were formed after 9 days of suspension culture (FIG. 9A) and transferred to plates coated with 5 ng/ml Fibronectin (Sigma). Attached cells were cultured for 6-9 days, and then used for further analysis about the expression of several differentiation markers detected by RT-PCR. AFP, CK19, Runx2, GATA2, Oligo2 were found highly expressed in EB derived from iPS cells and ES cells, compared to their low expression in fibroblasts (FIG. 9B). For neural cell differentiation, the 4-day EBs were plated in chemical defined medium (DMEM/F12 with N2B27 supplement, Invitrogen) on matrigel-coated plates. For mesoderm induction, iPS cells were passaged onto matrigel-coated plastic plates and cultured in MEF-conditioned medium. On the next day after passage, differentiation was carried out in serum-free medium (SFM): RPMI 1640 (HyClone) supplemented with 1% Insulin-Transferrin-Selenium-A (Invitrogen), 0.1 mM NEAA (Invitrogen), 2 mM L-Glutamine, 0.1 mM 2-ME, 100 U/ml penicillin, 100 μg/ml streptomycin, and supplemented with 25 ng/ml BMP-4 (Peprotech) and 50 ng/ml bFGF (Zhang et al., 2008). For endoderm differentiation, iPS cells were cultured in 1640 medium (Hyclone) supplemented with 0.5 mg/ml albumin fraction V (Sigma-Aldrich) and 100 ng/ml Activin A (Peprotech) for 1 day. On the following 2 days, 1% insulin-transferrinselenium (ITS) (Sigma-Aldrich) was added to this medium. After 3 days of Activin A treatment, the differentiated cells were cultured in hepatocyte culture medium (HCM) (Cambrex, Baltimore, Md.) supplemented with 30 ng/ml FGF4 (Peprotech) and 20 ng/ml BMP2 (Peprotech) for 5 days. Then the differentiated cells were further maturated in HCM supplemented with 20 ng/ml HGF (Peprotech) for 5 days, and 10 ng/ml OSM (Peprotech) plus 0.1 μM Dex (Tocris) from then on. For trophoblast differentiation, iPS cells and hES cells were plated onto matrigel coated dish, and treated by BMP4 for nine days (Xu et al., 2002). The 24 h culture medium supernatant in 3 parallel experiments were mixed and analyzed by human chorionic gonadotropin (hCG) ELISA (Bio-quant) (FIG. 9D). The medium was changed every day.

### 7. Teratoma Formation

2-5×10⁶ iPS cells were injected into the kidney capsule of a SCID beige mouse (Weitongda Biotech). Teratomas were recovered 6-9 weeks after grafting. Control mice were injected with 10 million hAFF and hFSF fibroblasts and failed to form teratoma (three injections in total). Then they were embedded in paraffin and processed with hematoxylin and eosin staining and immunohistochemistry staining, the primary antibodies, monoclonal mouse anti-α-SMA (Millipore, dilution 1:200), anti-CK 19 (Dako, 1:100), anti-VIMENTIN (Dako, 1:200), anti-NEUROFILAMENT (Santa cruz, 1:200), and polyclonal rabbit anti-β-III-TUBULIN (Sigma, 1:200) were applied and incubated in a moist chamber at 4° C. overnight. Immunodetection was performed using the Anti-Mouse and the Anti-Rabbit Non-Biotin HRP Detection System (Zymed Laboratories) (FIG. 2E).

### 8. Bisulfite Sequencing

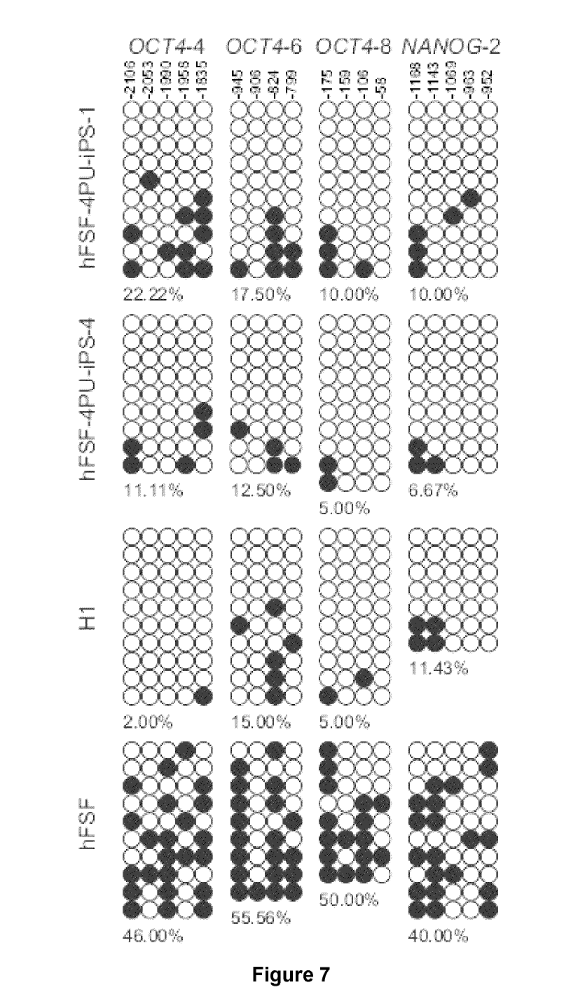

Genomic DNA was bisulfite converted by the CpGenome™ DNA modification kit (Intergen) according to the manufacturer's protocol. Modified DNA was amplified by PCR. Primer sequences are shown in Table 4. (SEQ ID NO: 84-91). PCR products were sequenced (FIG. 7).

### 9. DNA Microarray

Total mRNA from hFSF, hAFF, hES cells (H1, H7) and the established cell lines were labeled with Cy5, hybridized to a human Oligo Microarray (Phalanx Human Whole Genome OneArray™, Phalanx Biotech) according to the manufacturer's protocol. Three technical repetitions were performed. After hybridization, arrays were scanned using GenePix 4000B scanner (Molecular Devices) and processed using the GenePix Pro 6.0 software (Molecular Devices). After removing control probes, a 14/33 presence call (SNR>=5 and foreground-background>0) was used to filter probes for the 33 microarrays, resulting in 12733 probes for further quantile normalization. The median of each sample was used for hierarchical clustering. Complete-linkage hierarchical clustering was performed using cluster 3.0 (written by Michael Eisen, Stanford University) with Spearman rank correlation coefficient as gene distances measurement and Pearson correlation coefficient as sample distances measurement (FIG. 2C).

### Results:

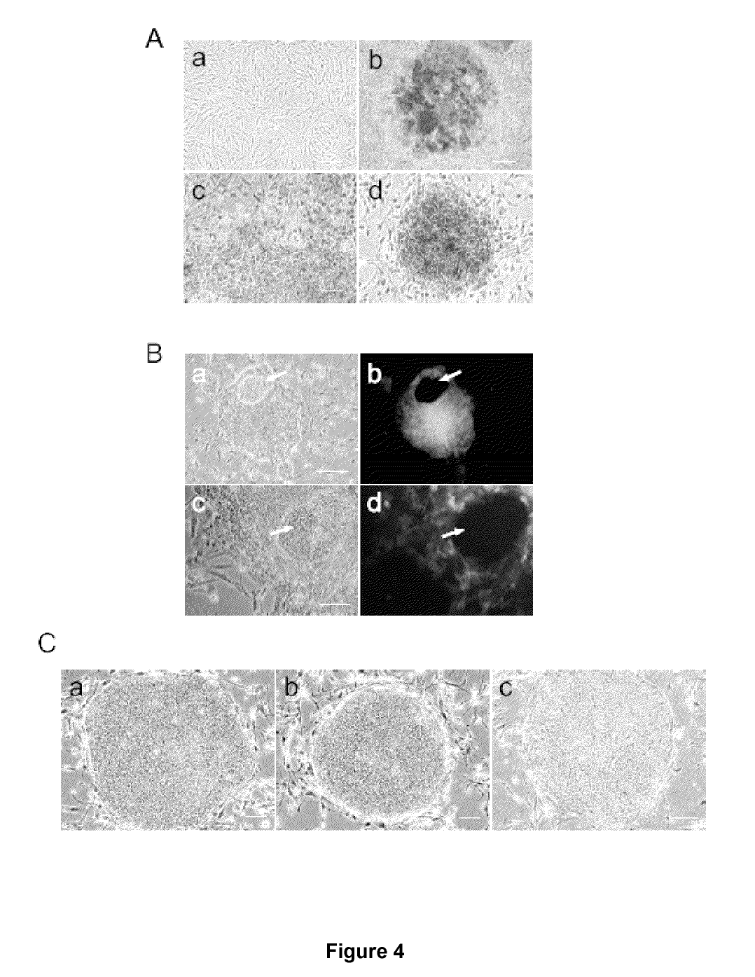

To increase the efficiency of human iPS cell generation, we screened a panel of candidate factors (including NANOG, LIN28, DPPA4, DPPA5, ZIC3, BCL-2, h-RAS, TPT1, SALL2, NAC1, DAX1, TERT, ZNF206, FOXD3, REX1, UTF1 and p53-siRNA) that might facilitate the reprogramming process. The reported four human reprogramming factors (OCT4, SOX2, KLF4 and c-MYC) (Lowry et al., 2008; Park et al., 2008; Takahashi et al., 2007) along with individual additional candidate factors, were cloned into the lentiviral vector pLL3.7-ΔU6 (FIG. 3A), and the oligo DNA for p53 shRNA (Brummelkamp et al., 2002) and a nonsense shRNA were cloned into the lentiviral vector pLL3.7 (FIG. 3B). The lentivirus transduction efficiency was very high, as indicated by the expression of EGFP and the introduced genes in the pLL3.7 vector (FIG. 3C)□and the knockdown of p53 expression by p53 shRNA was efficient (FIG. 3). In our experiment, when OCT4, SOX2, KLF4 and c-MYC were introduced into fibroblasts from the foreskin of an adult (hAFF), cell clusters started to emerge at day 10 post-transduction. When analyzed by AP staining at day 18 post-transduction, the colonies were divisible into 3 groups: AP-negative granular colonies, AP-low/noncompact colonies and highly AP-positive colonies, which exhibited a compact morphology and more closely resembled human embryonic stem (hES) cells (FIG. 4A). Subsequently, the additional candidate factors were separately introduced into fibroblasts together with the reported four reprogramming factors (referred to as the 4+1 strategy). 18 days post-transduction, cells that had been introduced with 4+p53 siRNA and 4+UTF1 exhibited an increased efficiency in the yield of highly AP-positive colonies (FIG. 1A). A nonsense siRNA was used as a control for the p53 siRNA, and no effect on increasing the efficiency of AP-positive colony generation was found. Strikingly, when these six factors (4+p53 siRNA+UTF1) were used together, the efficiency of highly AP-positive colony generation was elevated by more than 200 times compared with just using the four factors (FIG. 1A). Moreover, when the six factors were introduced into different kinds of target cells, including neonatal fibroblasts (BJ fibroblasts), human embryonic fibroblasts (hEF) and fetal skin fibroblasts (hFSF), highly AP-positive colonies were also generated at a comparably high efficiency. Similar effects were also found when the retroviral vector pMX was used, even in monkey skin fibroblasts or mouse embryonic fibroblasts.

Based on the observation that AP activity was only an early event in mouse iPS cell generation (Brambrink et al., 2008; Stadtfeld et al., 2008), we further investigated whether p53 siRNA and UTF1 would enhance the efficiency of generating final stage iPS cells from human adult fibroblasts. We found that before day 18 post-transduction, the highly AP-positive colonies were mostly GFP-positive (GFP was driven by the CMV promoter in the lentiviral vector pLL3.7, and was used as an indicator of exogenous transgenes expression, FIG. 1Bh and FIG. 3B). When several GFP positive colonies were chosen for further analyses, they maintained AP activity and GFP expression for at least 20 passages, but displayed an extremely low efficiency of spontaneous and directed differentiation in vitro, and none of them could form teratoma with cells of all 3 germ layers in vivo, although some of them could differentiate into a certain lineage. These results suggest that the GFP-positive cells might be partially reprogrammed cells of iPS cell generation (referred to as pre-iPS cells). RT-PCR and Microarray data further confirmed that these cells were trapped in an intermediate state (FIG. 2 and FIG. 6).

After day 18 post-transduction, GFP silenced colonies began to emerge (FIG. 1Bd and FIG. 4B), which had a lighter appearance than GFP-positive cells under phase contrast microscopy, and more closely resembled hES cell colonies in morphology, with clear-cut round edges, high nucleus/cytoplasm ratio, homogeneity and a tendency to form monolayers (FIG. 1B). Based on previous published studies suggesting that exogenous gene silencing might distinct a pluripotency state (Chung et al., 2002; Hong et al., 2007; Hotta and Ellis, 2008; Nakagawa et al., 2008; Stadtfeld et al., 2008; Xia et al., 2007), we calculated the number of iPS cell colonies by using exogenous GFP silencing and morphology resembling hES cells as the criteria. On day 25 post-transduction, we found that 4+p53 siRNA and 4+UTF1 significantly increased the efficiency of iPS cell generation (FIG. 1Cc). Moreover, when the six factors were used, the number of generated iPS cell colonies was elevated up to 100 times, compared with just using the four factors (FIG. 1Cc). Interestingly, we found that p53 siRNA dramatically enhanced the total numbers of cell colonies, but it did not further exert its effect on the highly AP-positive colony numbers or iPS cell numbers (FIG. 1C). Meanwhile, UTF1 had little effect on the total colony numbers, but it significantly increased the highly AP-positive colony numbers and iPS colony numbers (FIG. 1C). This suggests that p53 siRNA and UTF1 have effects at different stages and can synergize in support of iPS cell generation. A similar effect was also achieved when other fibroblasts were used, including BJ, hFSF and hEF (FIG. 4C). To confirm the iPS generation efficiency estimated by using exogenous GFP silencing and morphology resembling hES cells as the criteria, we performed immunostaining for the endogenous expression of NANOG and TRA-1-81, whose activation was considered as a characteristic of iPS cells (Brambrink et al., 2008; Huangfu et al., 2008; Okita et al., 2007; Shi et al., 2008; Stadtfeld et al., 2008; Wernig et al., 2007), on the primary culture dish to generate iPS cells. We found that all the colonies identified by GFP-/ES-like were positive for NANOG and TRA-1-81 (FIG. 5). The number of colonies counted by GFP-/ES-like was very similar to that judged by the expression of NANOG and TRA-1-81 (FIG. 5). Moreover, all of the colonies chosen based on our criteria were further validated to be iPS cells (further validation of these cells is provided in Table 1). Taken together, the results demonstrate that p53 siRNA and UTF1 were not only able to dramatically increase the efficiency of generating highly AP-positive colonies, but also generating fully reprogrammed iPS cells by up to 100 folds.

We also determined whether UTF1 and p53 siRNA could substitute for the function of the reported four reprogramming factors, by withdrawing each one of them individually and using the remaining five factors to induce iPS cells from hAFF cells (6-1 strategy). We found that when OCT4, SOX2 or KLF4 were separately removed from the six factors (6-OCT4,6-SOX2, and 6-KLF4), although highly AP-positive colonies were obtained (FIG. 1Db), no iPS cell colonies were found, even extending the culture period to 60 days post-transduction. These results suggest the essential role of OCT4, SOX2 or KLF4 to fulfill the iPS cell induction process. When c-MYC was removed (6-MYC), the total colony number and highly AP-positive colony number decreased dramatically (FIG. 1Da and FIG. 1Db). However, during further cultivation, cell colonies continuously emerged and subsequently developed into iPS cell colonies. On day 35 post-transduction, the efficiency of iPS induction by 6-MYC was 71-223 iPS cell colonies from 1×10⁵ target cells, which was comparable to that using the six factors (FIG. 1Dc). Interestingly, when c-MYC was removed (6-MYC), the colonies which emerged in further cultivation were mostly iPS colonies, compared with the large quantity of incompletely reprogrammed cell colonies when the six factors were used (FIG. 1D and FIG. 1E), which was also described by (Nakagawa et al., 2008). Taken together, these data suggest that although c-MYC benefits cell colony origination, it is not indispensable in the iPS cell generation process, especially in the presence of p53 siRNA and UTF1.

Previous reports (Nakagawa et al., 2008; Wernig et al., 2008) and our data (FIG. 1E) demonstrated that when c-MYC was withdrawn from the reported four reprogramming factors, the efficiency of generating human iPS cell decreased dramatically compared with using the four factors. Notably, our data show that when p53 siRNA and UTF1 were added together to the combination of 4-MYC (OCT4, SOX2 and KLF4), the efficiency of iPS cell generation increased more than 100 folds (FIG. 1Dc and FIG. 1Ec). We further studied whether p53 siRNA or UTF1 alone had a similar effect. We found that compared with 4-MYC, the addition of p53 siRNA (4-MYC+p53 siRNA) or UTF1 (4-MYC+UTF1) dramatically increased the efficiency of iPS cell generation (FIG. 1Ec). In particular, when the combination of OCT4, SOX2, KLF4 and UTF1 was used (4-MYC+UTF1), iPS cells were induced at efficiency of approximately 3×10⁻⁴, which was at least 10 times higher than 4-MYC (FIG. 1Ec). Moreover, iPS cell colonies can be successfully obtained every time in our experiment by using 4-MYC+UTF1, compared with that only in half of the experiments iPS cells could be obtained by using 4-MYC, which was similar to the previous report by (Nakagawa et al., 2008). Therefore, by replacing c-MYC with UTF1 from the reported four reprogramming factors, we have identified a novel combination of 4 reprogramming factors to generate iPS cells (OCT4, SOX2, KLF4 and UTF1), which is more efficient, more reproducible and dispensable for the oncogene c-MYC. In addition, the ratio of iPS cell colonies to total colonies was much higher when the new combination of four factors was used (FIG. 1C and FIG. 1E), which made a more specific induction from fibroblasts to iPS cells.

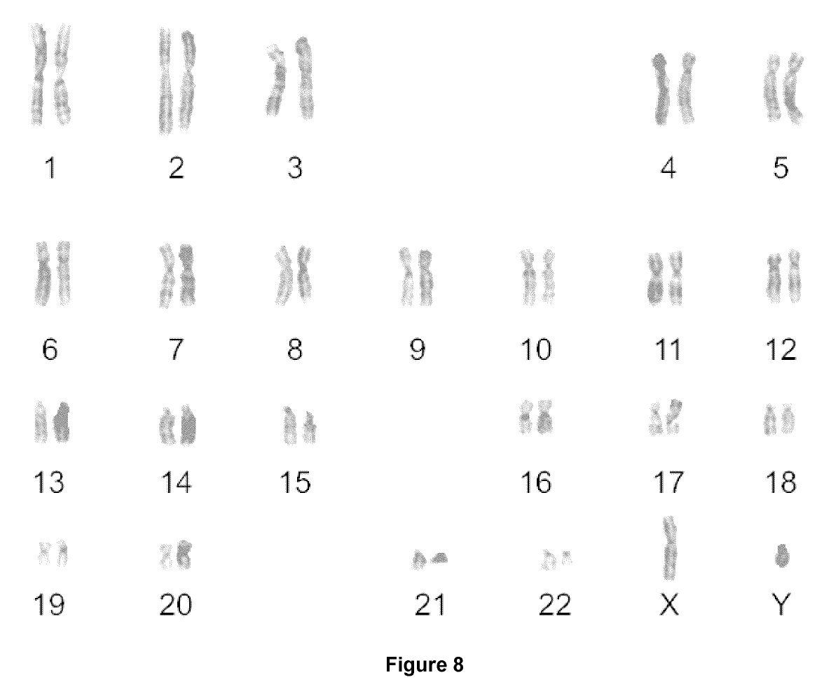

We established a panel of human iPS cell lines from fibroblasts using different combinations of reprogramming factors. These cell lines were further characterized for plutipotency, gene expression profile, differentiation potential, and genomic stability (Table 1). The representative data are shown in FIG. 2-9. RT-PCR analysis indicated that the endogenous pluripotency transcription factors (OCT4, SOX2 and UTF1), and other pluripotency marker genes (LIN28, SALL4, NODAL, TDGF1, OTX2, ZNF206, LEFTY1, LEFTY2, DPPA4, SALL2, NANOG and DNMT3b), were expressed at levels similar to those of hES cells (FIG. 2A). It also showed that the expression of exogenous genes in most of the iPS cell lines have been silenced (FIG. 6), which is consistent with GFP silencing, and further suggest the pluripotent state (Hotta and Ellis, 2008; Stadtfeld et al., 2008; Xia et al., 2007). We also found that in iPS cells generated with p53 siRNA introduction, the expression level of p53 was much higher than in pre-iPS cells, suggesting the attenuation of p53 siRNA in the fully reprogrammed iPS cells. Subsequently, the expression of SSEA4, TRA1-60, TRA1-81 and NANOG was detected by immunostaining (FIG. 2B). Microarray data also uncovered a similar gene expression profile pattern between iPS cells and hES cells (FIG. 2C). Bisulfite sequencing further revealed that OCT4 and NANOG promoters were demethylated in iPS cells, which was similar to hES cells and different from fibroblasts (FIG. 7). Chromosomal G-band analysis showed that the iPS cells exhibited a normal karyotype (FIG. 8). Moreover, the human iPS cell lines further analyzed in our work were stable in culture. So far, several lines, generated by UTF1 and P53 siRNA introduction, have been passaged for more than 5 months. STR analysis showed that these cells were derived from parental fibroblasts, not by contamination from existing hES cells in our laboratory (Table 2).

In order to analyze the differentiation potential of iPS cell lines, we tested their capacity for spontaneous and direct differentiation into the cell types of the three germ layers. After 8 days of floating cultivation, embryoid bodies (EB) were formed (FIG. 9A). They were then replated and attached to gelatin-coated plates and cultured for another 8 days (Takahashi et al., 2007). Immunocytochemical staining showed that the attached cells were positive for NEUROFILAMENT (ectoderm), α-SMA (mesoderm), VIMENTIN (mesoderm and partial endoderm) and ALBUMIN (endoderm) (FIG. 2D). As controls, fibroblasts and undifferentiated iPS cells did not express these markers. RT-PCR analysis also confirmed the results (FIG. 9B). Moreover, iPS cells were induced to differentiate directly into neural cells (Yao et al., 2006). The differentiated cells expressed the neural markers, PAX6, NEUROFILAMENT, B-III-TUBULIN (FIG. 2D) and NESTIN. Directed differentiation to hematopoietic lineage cells (Zhang et al., 2008) resulted in BRACHYURY expression (FIG. 2D), and subsequent GATA2, CD31, KDR expression (FIG. 9C). Directed differentiation to endoderm cell lineages was also performed as reported previously (Cai et al., 2007; Jiang et al., 2007). The differentiated human iPS cells were positive for CK19, ALB, SOX17, FOXA2, AFP (FIG. 2D), CK8, CK18 and PDX1. When induced with BMP4 for nine days and detected by human chorionic gonadotropin (hCG) ELISA, the human iPS cells exhibited differentiation potential towards trophoblasts (FIG. 9D), which is similar to hES cells (Xu et al., 2002). Moreover, five iPS cell lines were chosen for teratomas test, and all of them were found to form teratomas in vivo 6-9 weeks after injection (Table 1). In these teratomas, tissues of all three germ layers were found (FIG. 2E). In conclusion, the iPS cells generated in this work have a similar gene expression profile and differentiation potential as human embryonic stem (hES) cells.

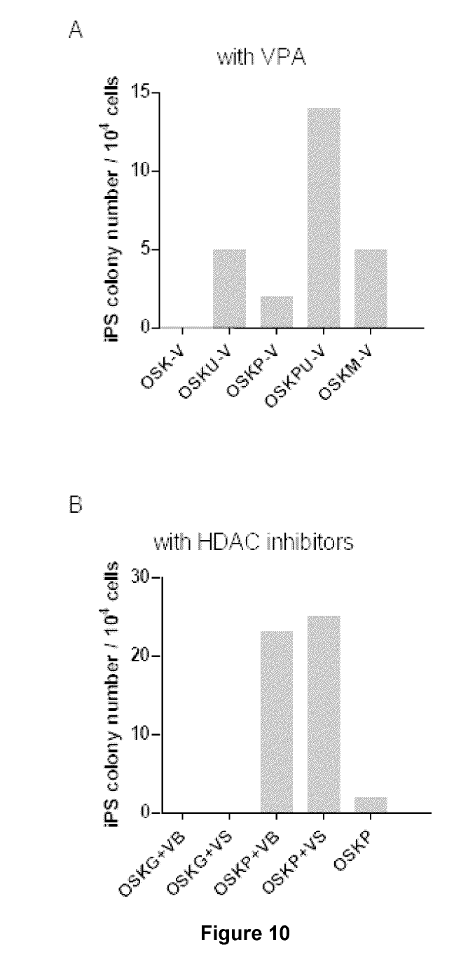

Recently, it is reported that iPS cells could be generated when contacting with HDAC inhibitors (and/or other compounds) by a much higher efficiency. We further tested whether UTF1 and p53 siRNA would still work in the context of HDAC inhibitors. We found that when VPA was added in the reprogramming process, UTF1 or/and p53 siRNA could still enhance the reprogramming efficiency significantly (FIGS. 10A and 10B).

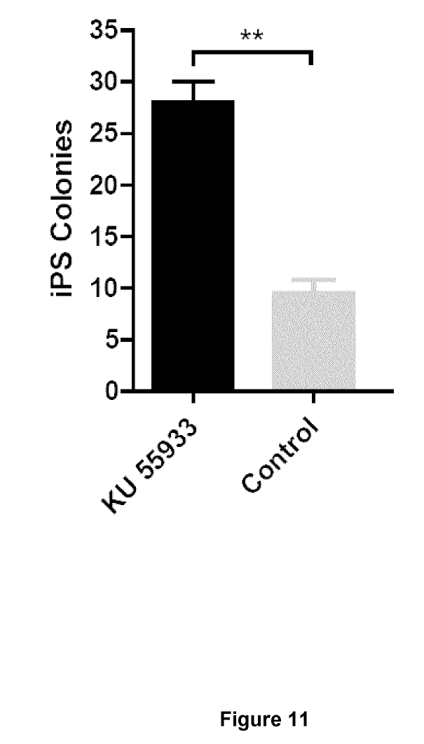

We also addressed whether p53 inactivation could enhance reprogramming by means other than RNA interference. A chemical compound named KU55933 (Tocris) was used, which could selectively inhibit p53 pathway. As expected, KU55933 improved reprogramming efficiency by almost 3 times when oct4, sox2 and Klf4 were introduced into mouse embryonic fibroblasts, as shown in FIG. 11.

In summary, we have identified two factors, p53 inactivation and UTF1, that can significantly improve the reprogramming process.

### Tables:

TABLE 1
Characterization of established iPS clones

| | | | | | | Differentiation | | | |
|---|---|---|---|---|---|---|---|---|---|
| | | | Gene Expression profile | | | potential | | Others | |
| Clone | Source | Factors | RT-PCR | IHC | Microarray | In vitro | Teratoma | Karyotype | STR |
| 1 | hFSF | OSMKPU | ✓ | ✓ | ✓ | ✓ | ✓ | ✓(7.15) | ✓ |
| 2 | | | ✓ | ✓ | ✓ | ✓ | | | |
| 4 | | | ✓ | ✓ | ✓ | ✓ | ✓ | | ✓ |
| 12 | hEF | OSMKPU | ✓ | ✓ | | ✓ | | | ✓ |
| 16 | | | ✓ | | | | | | |
| 1 | BJ | OSKPU | ✓ | ✓ | | | | | ✓ |
| 2 | | | ✓ | ✓ | | | | | |
| 1 | hAFF | OSMKPU | ✓ | ✓ | ✓ | ✓ | ✓ | ✓(31) | ✓ |
| 9 | | | ✓ | ✓ | ✓ | ✓ | ✓ | | |
| 13 | | | ✓ | ✓ | ✓ | ✓ | ✓ | | |
| 21 | | | ✓ | ✓ | | | | | |
| 2 | | OSKPU | ✓ | ✓ | ✓ | ✓ | | ✓(25) | ✓ |
| 4 | | | ✓ | ✓ | ✓ | ✓ | | | ✓ |
| 5 | | | ✓ | ✓ | ✓ | ✓ | | | |
| 6 | | | ✓ | ✓ | | ✓ | | | |
| 8 | | | ✓ | ✓ | | ✓ | | | |
| 1 | | OSKU | ✓ | ✓ | ✓ | ✓ | ✓ | ✓(13) | ✓ |
| 3 | | | ✓ | ✓ | ✓ | ✓ | ✓ | ✓(22) | ✓ |
| 4 | | | ✓ | ✓ | | ✓ | | | |
| 7 | | | ✓ | ✓ | | ✓ | | | |
| 8 | | | ✓ | ✓ | | ✓ | | | |
| 1 | | OSK | ✓ | ✓ | | ✓ | | | ✓ |
| 2 | | | ✓ | ✓ | | ✓ | | | ✓ |
| 3 | | | ✓ | ✓ | | | | | |
| 4 | | | ✓ | ✓ | | | | | |
| 1 | | OSMK | ✓ | ✓ | | ✓ | | | |

IHC: Immunohistochemistry,
STR; Short-tandem repeat analysis;
Passage numbers when karyotype analysis was performed were shown in parenthesis.

TABLE 2
STR results

| | | | | | | | | | hAFF-4U- | | hFSF- | | hFSF- | | hEF-4PU- | |
|---|---|---|---|---|---|---|---|---|---|---|---|---|---|---|---|---|
| Locus/Clone | BJ | | hAFF | | hEF | | hFSF | | M-iPS-3 | | 4PU-iPS-1 | | 4PU-iPS-4 | | iPS-12 | |
| D3S1358 | 14 | 16 | 16 | 17 | 15 | 18 | 15 | 17 | 16 | 17 | 15 | 17 | 15 | 17 | 15 | 18 |
| TH01 | 7 | 8 | 9 | 9 | 7 | 7 | 6 | 8 | 9 | 9 | 6 | 8 | 6 | 8 | 7 | 7 |
| D21S11 | 29 | 29 | 30 | 32.2 | 28 | 32.2 | 28 | 32.2 | 30 | 32.2 | 28 | 32.2 | 28 | 32.2 | 28 | 32.2 |
| D18S51 | 17 | 19 | 18 | 19 | 13 | 15 | 16 | 18 | 18 | 19 | 16 | 18 | 16 | 18 | 13 | 15 |
| PentaE | 7 | 17 | 10 | 18 | 12 | 13 | 10 | 12 | 10 | 18 | 10 | 12 | 10 | 12 | 12 | 13 |
| D5S818 | 12 | 12 | 10 | 12 | 11 | 12 | 11 | 11 | 10 | 12 | 11 | 11 | 11 | 11 | 11 | 12 |
| D13S317 | 8 | 9 | 10 | 10 | 8 | 11 | 8 | 8 | 10 | 10 | 8 | 8 | 8 | 8 | 8 | 11 |
| D7S820 | 11 | 12 | 11 | 11 | 11 | 12 | 10 | 11 | 11 | 11 | 10 | 11 | 10 | 11 | 11 | 12 |
| D16S539 | 9 | 13 | 9 | 10 | 9 | 10 | 8 | 9 | 9 | 10 | 8 | 9 | 8 | 9 | 9 | 10 |
| CSF1P0 | 10 | 12 | 11 | 12 | 11 | 13 | 10 | 11 | 11 | 12 | 10 | 11 | 10 | 11 | 11 | 13 |
| PentaD | 12 | 13 | 14 | 14 | 9 | 10 | 12 | 13 | 14 | 14 | 12 | 13 | 12 | 13 | 9 | 10 |
| Amelogenin | X | Y | X | Y | X | X | X | Y | X | Y | X | Y | X | Y | X | X |
| vWA | 16 | 18 | 16 | 20 | 14 | 17 | 14 | 17 | 16 | 20 | 14 | 17 | 14 | 17 | 14 | 17 |
| D8S1179 | 9 | 11 | 12 | 16 | 13 | 15 | 8 | 15 | 12 | 16 | 8 | 15 | 8 | 15 | 13 | 15 |
| TPOX | 10 | 11 | 8 | 8 | 8 | 9 | 8 | 11 | 8 | 8 | 8 | 11 | 8 | 11 | 8 | 9 |
| FGA | 22 | 23 | 24 | 25 | 24 | 24 | 22 | 23 | 24 | 25 | 22 | 23 | 22 | 23 | 24 | 24 |

| | | | | | |
|---|---|---|---|---|---|
| Locus/Clone | HE-1* | HE-2* | H1 | H7 | H9 |

| | | | | | | | |
|---|---|---|---|---|---|---|---|
| D3S1358 | 16 | 20 | 15 | 17 | ND | ND | ND |

| | | | | | | | | | | |
|---|---|---|---|---|---|---|---|---|---|---|
| TH01 | 9 | 9 | 9 | 9 | 9 | 3 | 6 | 6 | 9 | 3 |

| | | | | | | | |
|---|---|---|---|---|---|---|---|
| D21S11 | 29 | 32.2 | 29 | 31 | ND | ND | ND |
| D18S51 | 13 | 13 | 13 | 13 | ND | ND | ND |
| PentaE | 11 | 14 | 11 | 14 | ND | ND | ND |

| | | | | | | | | | | |
|---|---|---|---|---|---|---|---|---|---|---|
| D5S818 | 11 | 12 | 10 | 10 | 9 | 11 | 11 | 13 | 11 | 12 |
| D13S317 | 11 | 12 | 11 | 12 | 8 | 11 | 11 | 12 | 9 | 9 |
| D7S820 | 10 | 11 | 10 | 11 | 8 | 12 | 10 | 11 | 9 | 11 |
| D16S539 | 9 | 9 | 9 | 9 | 9 | 13 | 12 | 13 | 12 | 13 |
| CSF1P0 | 11 | 12 | 12 | 12 | 12 | 13 | 12 | 12 | 11 | 11 |

| | | | | | | | |
|---|---|---|---|---|---|---|---|
| PentaD | 12 | 12 | 12 | 14 | ND | ND | ND |

| | | | | | | | | | | |
|---|---|---|---|---|---|---|---|---|---|---|
| Amelogenin | X | Y | X | X | X | Y | X | X | X | X |
| vWA | 14 | 15 | 14 | 15 | 15 | 17 | 14 | 15 | 17 | 17 |

| | | | | | | | |
|---|---|---|---|---|---|---|---|
| D8S1179 | 13 | 14 | 13 | 13 | ND | ND | ND |

| | | | | | | | | | | |
|---|---|---|---|---|---|---|---|---|---|---|
| TPOX | 8 | 9 | 8 | 9 | 8 | 11 | 8 | 11 | 10 | 11 |

| | | | | | | | |
|---|---|---|---|---|---|---|---|
| FGA | 24 | 26 | 23 | 24 | ND | ND | ND |

*Two hES cell lines estabilished in our lab

TABLE 3
Primers for human gene construction

| | | | |
|---|---|---|---|
| | | ACCESSION | |
| NO. | SYMBOL | NUMBERS | PRIMERS FOR CLONING |

| | | | |
|---|---|---|---|
| 1 | KLF4 | NM_004235 | TACTCGAGGCCACCATGGCTGTCAGCGACGC |
| | | | GGCGAATTCATTAAAAATGCCTCTTCATGTG |
| 2 | SOX2 | NM_003106 | CCTCGAGCCACCATGTACAACATGATGGAG |
| | | | CGGAATTCATCACATGTGTGAGAGAGGGGC |
| 3 | CMYC | NM_002467 | ACTCGAGCCACCATGCCCCTCAACGTTAGC |
| | | | CGGAATTCATTACGCACAAGAGTTCCGTAG |
| 4 | POU5F1 | NM_002701 | AGGATCCGCCACCATGGCGGGACACCTGGC |
| | | | GCGAATTCATCAGTTTGAATGCATGGGAGG |
| 5 | UTF1 | NM_003577 | AACTCGAGCCACCATGCTGCTCCGGCCCCGC |
| | | | AAGAATTCCACTGGCACGGGTCCCTGAGGA |
| 6 | REX1 | NM_020695 | AACTCGAGCCACCATGAGCCAGCAACTGAAGAAAC |
| | | | TTGATATCCTACTTTCCCTCTTGTTCATTC |
| 7 | NANOG | NM_024865 | AACTCGAGCCACCATGAGTGTGGATCCAGCTTG |
| | | | CCGAATTCATCACACGTCTTCAGGTTGCATGTTC |
| 8 | BCL2 | NM_000633 | ACTCGAGCCACCATGGCGCACGCTGGGAGAAC |
| | | | ACGAATTCTCACTTGTGGCCCAGATAGG |
| 9 | DAX1 | NM_000475 | TCTCGAGCCACCATGGCGGGCGAGAACCACCA |
| | | | ACGAATTC TTATATCTTTGTACAGAGCA |
| 10 | DPPA4 | NM_018189 | ACTCGAGCCACCATGTTGCGAGGCTCCGCTT |
| | | | CCGAATTCACTATTCCCATTGGAGGCT |
| 11 | DPPA5 | NM_001025290 | TAGCTAGCCACCATGGGAACTCTCCCGGCA |
| | | | GCGAATTCATCACTTCATCCAAGGGCCTAG |
| 12 | FOXD3 | NM_012183 | AACTCGAGCCACCATGACCCTCTCCGGCGGC |
| | | | GGGAATTCACTATTGCGCCGGCCATTTG |
| 13 | HESX1 | NM_003865 | ACTCGAGGCCACCTGTCTCCCAGCCTTCAGGAA |
| | | | CGAATTCCTATTCCAGCAGATTTGTGTTG |
| 14 | HRAS | NM_005343 | ACTCGAGCCACCATGACGGAATATAAGCTGGT |
| | | | CGAATTCTCAGGAGAGCACACACTTGCAG |
| 15 | TERT | NM_195255 | ATGCCGCGCGCTCCCCGCTG |
| | | | TCAGTCCAGGATGGTCTTGAAGT |
| 16 | LIN28 | NM_024674 | ACTCGAGCCACCATGGGGCTCCGTGTCCAAC |
| | | | CCGAATTCATCAATTCTGTGCCTCCGGGA |
| 17 | NAC1 | NM_052876 | TACTCGAGGCCACCATGGCCCAGACACTGCAGAT |
| | | | GCCGAATTCATTACTGCAGGGCTTCAGCCGAGG |
| 18 | SALL2 | NM_005407 | CTCGAGCCACCATGTCTCGGCGAAAGCAG |
| | | | CCGATATCATTAAAACACCTTTAATGATAGAG |
| 19 | SET | NM_003011 | TCTCGAGCCACCATGTCGGCGCAGGCGGCCAA |
| | | | GGGAATTCATTAGTCATCTTCTCCTTCATCC |
| 20 | TPT1 | NM_003295 | CTCGAGCCACC ATGATTATCTACCGGGACCT |
| | | | CGAATTCCTCTCAAATGAGTTTAAATG |

TABLE 4
Primers for RT-PCR and bisulfite sequencing

| | | Product | |
|---|---|---|---|
| name | Sequences (5′-3′) | length | Application |
| pou5f1-F | GAA CCG AGT GAG AGG CAA CC | 457 | Endo Gene RT-PCR |
| pou5f1-R | ATC CCA AAA ACC CTG GCA CA | | |
| SOX2-F | ATG GGT TCG GTG GTC AAG TC | 299 | Endo Gene RT-PCR |
| SOX2-R | CCC TCC CAT TTC CCT CGT TT | | |
| c-MYC-F | GGA ACA AGA AGA TGA GGA AG | 610 | Endo Gene RT-PCR |
| c-MYO-R | TG ATT GCT CAG GAC ATT TC | | |
| KLF4-F | GCA AAA CCT ACA CAA AGA GT | 346 | Endo Gene RT-PCR |
| KLF4-R | GAC CAT GAT TGT AGT GCT TT | | |
| OCT4-Tg | GAA GGA TGT GGT CCG AGT G | 429 | Transgene RT-PCR |
| SOX2-Tg | CAT GGG TTC GGT GGT CAA | 388 | Transgene RT-PCR |
| c-MYC-Tg | TAC ATC CTG TCC GTC CAA GC | 311 | Transgene RT-PCR |
| KLF4-Tg | ACC ACT GTG ACT GGG ACG | 358 | Transgene RT-PCR |
| pLL-Tg | GCA GCG TAT CCA CAT AGC GT | | Transgene RT-PCR |
| Pou5f1-F | ATT CAG CCA AAC GAC CAT C | 485 | Total Gene RT-PCR |
| Pou5f1-R | GGA AAG GGA CCG AGG AGT A | | |
| SOX2-F | CAG CGC ATG GAC AGT TAC | 321 | Total Gene RT-PCR |
| SOX2-R | GGA GTG GGA GGA AGA GGT | | |
| c-MYC-F | AGT TTC ATC TGC GAC CCG | 439 | Total Gene RT-PCR |
| c-MYC-R | CCT CAT CTT CTT GTT CCT CCT | | |
| KLF4-F | GCG GGA AGG GAG AAG ACA | 384 | Total Gene RT-PCR |
| KLF4-R | CCG GAT CGG ATA GGT GAA | | |
| REX1-F | CCT AAA CAG CTC GCA GAA | 353 | Total Gene RT-PCR |
| REX1-R | CAG CCT TGA AAG GGA CAC | | |
| LIN28-F | GGG CAT CTG TAA GTG GTT | 483 | Total Gene RT-PCR |
| LIN28-R | GTA GGG CTG TGG ATT TCT | | |
| SALL4-F | TGA TGG GAG ACC AGG AGT | 488 | Total Gene RT-PCR |
| SALL4-R | GCG GGC TGA GTT ATT GTT | | |
| NODAL-F | ACA TCA TCC GCA GCC TAC A | 237 | Total Gene RT-PCR |
| NODAL-R | CCA TCT GAA ACC GCT CTA A | | |
| TDGF1-F | TCA GGA ATT TGC TCG TCC | 280 | Total Gene RT-PCR |
| TDGF1-R | CTT GGG CAG CCA GGT GT | | |
| OTX2-F | CCA CTT CGG GTA TGG ACT T | 260 | Total Gene RT-PCR |
| OTX2-R | TTG TTG GCG GCA CTT AGC T | | |
| ZNF206-F | TGG AGT GCC TGA CCT TTG | 460 | Total Gene RT-PCR |
| ZNF206-R | CGC ATG TGC AGC TTG AGA | | |
| LEFTY1-F | CAA CCG CAC CTC CCT CAT | 369 | Total Gene RT-PCR |
| LEFTY1-R | TTC TCG GCC CAC TTC ATC | | |
| LEFTY2-F | CAA CCG GAC CTC CCT CAT | 372 | Total Gene RT-PCR |
| LEFTY2-R | CAG TTC TTG GCC CAC TTC A | | |
| DPPA4-F | CCA ATC CCT CCA TTA CCT | 400 | Total Gene RT-PCR |
| DPPA4-R | CCC TGG CTG AAA TTC TCG | | |
| SALL2-F | GGC GAA AGC AGC GGA AAC | 383 | Total Gene RT-PCR |
| SALL2-R | TGT GGC AGC GAC GAG GAA | | |
| NANOG-F | TGC CTC AGA CGG AGA CTG | 353 | Total Gene RT-PCR |
| NANOG-R | GCT ATT CTT CGG CCA GTT | | |
| DNMT3B-F | GAC GAT GGC TAT CAG TCT TAC | 386 | Total Gene RT-PCR |
| DNMT3B-R | CCT ACC TTT ATG CCC AAC TC | | |
| GATA2-F | CCA CCC AAA GAA GTG TCT CCT | 461 | GATA2 RT-PCR |
| | GA | | |
| GATA2-R | CGG TTC TGC CCA TTG ATC TTG | | |
| SCL-F | TCT CTC GGC AGC GGG TTC TTT | 259 | SCL RT-PCR |
| SCL-R | CCA GGC GGA GGA TCT CAT TCT | | |
| | T | | |
| CD31-F | GGT GAC ACT GGA CAA GAA AGA | 218 | CD31 RT-PCR |
| CD31-R | GAC ATC GGA AGG ATA AAA CG | | |
| KDR-F | AGG GGC ACG ATT CCG TCA AG | 388 | KDR RT-PCR |
| KDR-R | TTT CAA AGG GAG GCG AGC AT | | |
| Oligo2-F | GCATCTGCCGAACCCAAGCAAT | 329 | Oligo2 RT-PCR |
| Oligo2-R | ACCCGCCAAGAAAGGAGGAAG | | |
| RUNX1-F | TGTGGTCCTATTTAAGCCAGCCCC | 312 | RUNX1 RT-PCR |
| RUNX1-R | TCAGGCTGGGCACGACGAATGCTC | | |
| AFP-F | AAATGCGTTTCTCGTTGC | 428 | AFP RT-PCR |
| AFP-R | CAGCCTCAAGTTGTTCCTCT | | |
| CK19-F | ACCAAGTTTGAGACGGAACAGGC | 469 | CK19 RT-PCR |
| CK19-R | TGTCTTCCAAGGCAGCTTTCATG | | |
| OCT4-4-F | GGA TGT TAT TAA GAT GAA GAT | | Bisulfite sequencing |
| | AGT TGG | | |
| OCT4-4-R | CCT AAA CTC CCC TTC AAA ATC | | |
| | TAT T | | |
| OCT4-6-F | GAA GGG GAA GTA GGG ATT ATT | | Bisulfite sequencing |
| | TTT | | |
| OCT4-6-R | CAA CAA CCA TAA ACA CAA TAA | | |
| | CCA A | | |
| OCT4-8-F | AAG TTT TTG TGG GGG ATT TGT | | Bisulfite sequencing |
| | AT | | |
| OCT4-8-R | CCA CCC ACT AAC CTT AAC CTC | | |
| | TA | | |
| NANOG-2-F | GAG TTA AAG AGT TTT GTT TTT | | Bisulfite sequencing |
| | AAA AAT TAT | | |
| NANOG-2-R | TCC CAA ATC TAA TAA TTT ATC | | |
| | ATA TCT TTC | | |

### REFERENCES

Aasen, T., A. Raya, et al. (2008). “Efficient and rapid generation of induced pluripotent stem cells from human keratinocytes.” Nat Biotechnol 26 (11): 1276-84.
Banito, A., S. T. Rashid, et al. (2009). “Senescence impairs successful reprogramming to pluripotent stem cells.” Genes Dev 23 (18): 2134-9.
Campbell, K. H., J. McWhir, et al. (1996). “Sheep cloned by nuclear transfer from a cultured cell line.” Nature 380 (6569): 64-6.
Carey, B. W., S. Markoulaki, et al. (2009). “Reprogramming of murine and human somatic cells using a single polycistronic vector.” Proc Natl Acad Sci USA 106 (1): 157-62.
Chen, S., Q. Zhang, et al. (2004). “Dedifferentiation of lineage-committed cells by a small molecule.” J Am Chem Soc 126 (2): 410-1.
Cowan, C. A., J. Atienza, et al. (2005). “Nuclear reprogramming of somatic cells after fusion with human embryonic stem cells.” Science 309 (5739): 1369-73.
Dimos, J. T., K. T. Rodolfa, et al. (2008). “Induced pluripotent stem cells generated from patients with ALS can be differentiated into motor neurons.” Science 321 (5893): 1218-21.
Feng, B., J. Jiang, et al. (2009). “Reprogramming of fibroblasts into induced pluripotent stem cells with orphan nuclear receptor Esrrb.” Nat Cell Biol 11 (2): 197-203.
Hong, H., K. Takahashi, et al. (2009). “Suppression of induced pluripotent stem cell generation by the p53-p21 pathway.” Nature 460 (7259): 1132-5.
Huangfu, D., R. Maehr, et al. (2008). “Induction of pluripotent stem cells by defined factors is greatly improved by small-molecule compounds.” Nat Biotechnol 26 (7): 795-7.
Huangfu, D., K. Osafune, et al. (2008). “Induction of pluripotent stem cells from primary human fibroblasts with only Oct4 and Sox2.” Nat Biotechnol 26 (11): 1269-75.
Jouneau, A. and J. P. Renard (2003). “Reprogramming in nuclear transfer.” Curr Opin Genet Dev 13 (5): 486-91.
Kawamura, T., J. Suzuki, et al. (2009). “Linking the p53 tumour suppressor pathway to somatic cell reprogramming.” Nature 460 (7259): 1140-4.
Kim, D., C. H. Kim, et al. (2009). “Generation of human induced pluripotent stem cells by direct delivery of reprogramming proteins.” Cell Stem Cell 4 (6): 472-6.
Kim, J. B., B. Greber, et al. (2009). “Direct reprogramming of human neural stem cells by OCT4.” Nature.
Kim, J. B., V. Sebastiano, et al. (2009). “Oct4-induced pluripotency in adult neural stem cells.” Cell 136 (3): 411-9.
Li, C., J. Zhou, et al. (2009). “Pluripotency can be rapidly and efficiently induced in human amniotic fluid-derived cells.” Hum Mol. Genet.
Li, H., M. Collado, et al. (2009). “The Ink4/Arf locus is a barrier for iPS cell reprogramming.” Nature 460 (7259): 1136-9.
Lyssiotis, C. A., R. K. Foreman, et al. (2009). “Reprogramming of murine fibroblasts to induced pluripotent stem cells with chemical complementation of Klf4.” Proc Natl Acad Sci USA 106 (22): 8912-7.
Maherali, N., R. Sridharan, et al. (2007). “Directly reprogrammed fibroblasts show global epigenetic remodeling and widespread tissue contribution.” Cell Stem Cell 1 (1): 55-70.
Mali, P., Z. Ye, et al. (2008). “Improved efficiency and pace of generating induced pluripotent stem cells from human adult and fetal fibroblasts.” Stem Cells 26 (8): 1998-2005.
Marion, R. M., K. Strati, et al. (2009). “A p53-mediated DNA damage response limits reprogramming to ensure iPS cell genomic integrity.” Nature 460 (7259): 1149-53.
Mikkelsen, T. S., J. Hanna, et al. (2008). “Dissecting direct reprogramming through integrative genomic analysis.” Nature 454 (7200): 49-55.
Nakagawa, M., M. Koyanagi, et al. (2008). “Generation of induced pluripotent stem cells without Myc from mouse and human fibroblasts.” Nat Biotechnol 26 (1): 101-6.
Okita, K., M. Nakagawa, et al. (2008). “Generation of mouse induced pluripotent stem cells without viral vectors.” Science 322 (5903): 949-53.
Park, I. H., N. Arora, et al. (2008). “Disease-specific induced pluripotent stem cells.” Cell 134 (5): 877-86.
Raya, A., I. Rodriguez-Piza, et al. (2009). “Disease-corrected haematopoietic progenitors from Fanconi anaemia induced pluripotent stem cells.” Nature 460 (7251): 53-9.
Shi, Y., C. Desponts, et al. (2008). “Induction of pluripotent stem cells from mouse embryonic fibroblasts by Oct4 and Klf4 with small-molecule compounds.” Cell Stem Cell 3 (5): 568-74.
Shi, Y., J. T. Do, et al. (2008). “A combined chemical and genetic approach for the generation of induced pluripotent stem cells.” Cell Stem Cell 2 (6): 525-8.
Silva, J., O. Barrandon, et al. (2008). “Promotion of reprogramming to ground state pluripotency by signal inhibition.” PLoS Biol 6 (10): e253.
Sommer, C. A., M. Stadtfeld, et al. (2009). “Induced pluripotent stem cell generation using a single lentiviral stem cell cassette.” Stem Cells 27 (3): 543-9.
Stadtfeld, M., M. Nagaya, et al. (2008). “Induced pluripotent stem cells generated without viral integration.” Science 322 (5903): 945-9.
Takahashi, K., K. Tanabe, et al. (2007). “Induction of pluripotent stem cells from adult human fibroblasts by defined factors.” Cell 131 (5): 861-72.
Takahashi, K. and S. Yamanaka (2006). “Induction of pluripotent stem cells from mouse embryonic and adult fibroblast cultures by defined factors.” Cell 126 (4): 663-76.
Utikal, J., N. Maherali, et al. (2009). “Sox2 is dispensable for the reprogramming of melanocytes and melanoma cells into induced pluripotent stem cells.” J Cell Sci 122 (Pt 19): 3502-10.
Utikal, J., J. M. Polo, et al. (2009). “Immortalization eliminates a roadblock during cellular reprogramming into iPS cells.” Nature 460 (7259): 1145-8.
Wernig, M., A. Meissner, et al. (2008). “c-Myc is dispensable for direct reprogramming of mouse fibroblasts.” Cell Stem Cell 2 (1): 10-2.
Yu, J., M. A. Vodyanik, et al. (2007). “Induced pluripotent stem cell lines derived from human somatic cells.” Science 318 (5858): 1917-20.
Zhao, Y., X. Yin, et al. (2008). “Two supporting factors greatly improve the efficiency of human iPSC generation.” Cell Stem Cell 3 (5): 475-9.
Zhou, H., S. Wu, et al. (2009). “Generation of induced pluripotent stem cells using recombinant proteins.” Cell Stem Cell 4 (5): 381-4.
Zhou, W. and C. R. Freed (2009). “Adenoviral Gene Delivery Can Reprogram Human Fibroblasts to Induced Pluripotent Stem Cells.” Stem Cells.
Brambrink, T., Foreman, R., Welstead, G. G., Lengner, C. J., Wernig, M., Suh, H., and Jaenisch, R. (2008). Sequential expression of pluripotency markers during direct reprogramming of mouse somatic cells. Cell Stem Cell 2, 151-159.
Cai, J., Zhao, Y., Liu, Y., Ye, F., Song, Z., Qin, H., Meng, S., Chen, Y., Zhou, R., Song, X., et al. (2007). Directed differentiation of human embryonic stem cells into functional hepatic cells. Hepatology 45, 1229-1239.
Hotta, A., and Ellis, J. (2008). Retroviral vector silencing during iPS cell induction: An epigenetic beacon that signals distinct pluripotent states. J Cell Biochem.
Jiang, W., Shi, Y., Zhao, D., Chen, S., Yong, J., Zhang, J., Qing, T., Sun, X., Zhang, P., Ding, M., et al. (2007). In vitro derivation of functional insulin-producing cells from human embryonic stem cells. Cell Res 17, 333-344.
Mali, P., Ye, Z., Hommond, H. H., Yu, X., Lin, J., Chen, G., Zou, J., and Cheng, L. (2008). Improved efficiency and pace of generating induced pluripotent stem cells from human adult and fetal fibroblasts. Stem Cells 26, 1998-2005.
Nakagawa, M., Koyanagi, M., Tanabe, K., Takahashi, K., Ichisaka, T., Aoi, T., Okita, K., Mochiduki, Y., Takizawa, N., and Yamanaka, S. (2008). Generation of induced pluripotent stem cells without Myc from mouse and human fibroblasts. Nat Biotechnol 26, 101-106.
Nishimoto, M., Fukushima, A., Okuda, A., and Muramatsu, M. (1999). The gene for the embryonic stem cell coactivator UTF1 carries a regulatory element which selectively interacts with a complex composed of Oct-3/4 and Sox-2. Mol Cell Biol 19, 5453-5465.
Rowland, B. D., Bernards, R., and Peeper, D. S. (2005). The KLF4 tumour suppressor is a transcriptional repressor of p53 that acts as a context-dependent oncogene. Nat Cell Biol 7, 1074-1082.
Stadtfeld, M., Maherali, N., Breault, D. T., and Hochedlinger, K. (2008). Defining Molecular Cornerstones during Fibroblast to iPS Cell Reprogramming in Mouse. Cell Stem Cell 2, 230-240.
van den Boom, V., Kooistra, S. M., Boesjes, M., Geverts, B., Houtsmuller, A. B., Monzen, K., Komuro, I., Essers, J., Drenth-Diephuis, L. J., and Eggen, B. J. (2007). UTF1 is a chromatin-associated protein involved in ES cell differentiation. J Cell Biol 178, 913-924.
Wernig, M., Meissner, A., Cassady, J. P., and Jaenisch, R. (2008). c-Myc is dispensable for direct reprogramming of mouse fibroblasts. Cell Stem Cell 2, 10-12.
Yamanaka, S. (2007). Strategies and new developments in the generation of patient-specific pluripotent stem cells. Cell Stem Cell 1, 39-49.
Zhao, R., and Daley, G. Q. (2008). From fibroblasts to iPS cells: Induced pluripotency by defined factors. J Cell Biochem.

What is claimed is:

1. A method for reprogramming a somatic cell, wherein the method comprises treating the somatic cell with factor A selected from one or more of the group consisting of Oct4, Klf4, Sox2, c-Myc, a MEK/ERK inhibitor, BIX01294, BAYK 8644, Kenpaullone, a GSK3 inhibitor, an HDAC inhibitor and a DNA methyl transferase inhibitor, and factor B selected from one or two of the group consisting of UTF1 and a p53 inactivation factor.

2. The method of claim 1, wherein the MEK/ERK inhibitor is PD0325901, the HDAC inhibitor is VPA, the GSK3 inhibitor is CHIR 99021, and the DNA methyl transferase inhibitor is 5-aza-C.

3. The method of claim 2, wherein one of Oct4(POU5f1), Sox2, c-Myc and Klf4 is not used.

4. The method of claim 3, wherein UTF1 and Oct4 are not used simultaneously; p53 inactivation factor and Klf4 are not used simultaneously.

5. The method of claim 4, wherein c-Myc is not used.

6. The method of claim 5, wherein the somatic cell is reprogrammed partially or completely.

7. The method of claim 6, wherein the somatic cell is reprogrammed completely into induced pluripotent stem cell.

8. The method of claim 7, wherein the somatic cell is obtained from human, mouse or primates and is a healthy cell or a cell containing at least one genetic lesion, and factor A selected from the group consisting of Oct4, Klf4, Sox2, and c-Myc and/or factor B are in the form of DNA, mRNA or proteins.

9. The method of claim 8, wherein the somatic cell is a fibroblast, a muscle cell, a cumulus cell, a neural cell, a liver cell, a GI tract cell, a mammary cell, a kidney cell, a blood cell, a vascular cell, a skin cell, an immune system cell, a lung cell, a bone cell, keratinocyte, or a pancreatic islet cell.

10. The method of claim 9, wherein the step of treating the somatic cell with factor A selected from one or more of the group consisting of Oct4, Klf4, Sox2 and c-Myc and factor B selected from one or two of the group consisting of UTF1 and a p53 inactivation factor comprises treating the somatic cell with one heterologous nucleic acid sequence encoding said factor A and said factor B, or the respective heterologous nucleic acid sequences encoding Oct4, Klf4, Sox2, c-Myc, UTF1 or a p53 inactivation factor, or the combinations thereof.

11. A method for improving the reprogramming efficiency of a somatic cell, the method comprising treating the somatic cell with factor B selected from one or two of the group consisting of UTF1 and a p53 inactivation factor.

12. A method for preparing a medicament for treating disease, characterized in that the usage of the cell produced by the method of claim 1, wherein the disease is selected from the group consisting of hematopoietic conditions, cardiac disorders and disorders such as liver disease, diabetes, thyroid abnormalities, neurodegenerative/neurological disorders, circulatory disorders, respiratory disorders, wound healing and/or repair, bone repair, and enzyme abnormalities.

13. A cell produced by a method of claim 1.

14. A kit for reprogramming a somatic cell, comprising factor A selected from one or more of the group consisting of Oct4, Klf4, Sox2, c-Myc, a MEK/ERK inhibitor, BIX01294, BAYK 8644, Kenpaullone, a GSK3 inhibitor, an HDAC inhibitor and a DNA methyl transferase inhibitor, as well as factor B selected from one or two of the group consisting of UTF1 and a p53 inactivation factor.

15. The kit of claim 14, wherein the MEK/ERK inhibitor is PD0325901, the HDAC inhibitor is VPA, the GSK3 inhibitor is CHIR 99021, and the DNA methyl transferase inhibitor is 5-aza-C.

16. A kit comprising: an iPS cell produced by a method of claim 1; at least one component for directing the iPS cell to a differentiated cell or expanding the iPS cell; and instructions.

17. The kit of claim 16, wherein the iPS cell is frozen or in culture.

18. The kit of claim 17, wherein the kit further comprises: a component for the detection of a marker for an iPS cell selected from a group selected from a group consisting of alkaline phophatase, NANOG, OCT4, SOX2, SSEA4, TRA-1-60 and TRA-1-81.

19. A composition comprising a more primitive precursor or a less differentiated cell compared to a somatic cell from which it was derived, and an exogenously produced substance selected from one or more of the group consisting of UTF1, p53 inactivation factor, Oct4, Klf4, Sox2, c-Myc, a MEK/ERK inhibitor, BIX01294, BAYK 8644, Kenpaullone, a GSK3 inhibitor, an HDAC inhibitor and a DNA methyl transferase inhibitor.

20. The composition of claim 19, wherein the MEK/ERK inhibitor is PD0325901, the GSK3 inhibitor is CHIR 99021, the HDAC inhibitor is VPA, and the DNA methyl transferase inhibitor is 5-aza-C.

21. The composition of claim 20, wherein the less differentiated cell is an iPS cell.

22. The composition of claim 21, wherein the iPS cell is produced by the method of claim 1.


SEQUENCE LISTING (91 sequences)

SEQ ID NO: 1 - DNA, 1083 bp, Artifical Sequence
  gene (1)..(1083): OCT4
atggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactga

SEQ ID NO: 2 - DNA, 954 bp, Artifical Sequence
  gene (1)..(954): SOX2
atgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtga

SEQ ID NO: 3 - DNA, 1320 bp, Artifical Sequence
  gene (1)..(1320): c-myc
atgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaa

SEQ ID NO: 4 - DNA, 1413 bp, Artifical Sequence
  gene (1)..(1413): KLF4
atggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaa

SEQ ID NO: 5 - DNA, 1026 bp, Artifical Sequence
  gene (1)..(1026): UTF1
atgctgctccggccccgcaggccgcccccgctcgcgccccccgcgccgccctcgcccgccagccccgaccccgagccgcggacacccggagacgccccggggaccccgccccggaggcccgcctcgcccagcgcgctgggggaactcgggttgccggtgtccccgggctcggcgcagcgcacgccctggagcgcccgggagacggagctgctgctggggacgctgctgcaaccggccgtgtggcgcgcgctgctcctggaccgccgccaggccctgcccacctaccgccgcgtgtcggccgcgctggcccagcagcaggtgcgccgcacccccgcgcagtgccgccgccgctacaagttccttaaagacaagtttcgcgaggcgcacggccagccgcccgggcccttcgacgagcagatccggaagctcatggggctgctgggcgacaacgggcgcaaacggcctcgccgccgctccccggggtccgggcgcccccagcgcgcccgccgcccggtccccaacgcgcacgcgccggctcccagcgaaccagacgccaccccgctgcccaccgcccgcgaccgcgacgcggaccccacctggacgctccgcttcagcccgtccccaccgaagtctgcggacgcctcccccgcccccggctccccgccagctcccgccccgaccgccctcgccacctgcatccccgaggaccgcgcgcccgtccgcggccccgggtccccgccgccacccccggcccgcgaagaccccgactcgccgcccggccgccccgaggactgcgcgccccctccggccgcgcccccgtcgctgaacaccgccctgctgcagaccctggggcacctgggcgacatcgcgaacatcctgggcccgctgcgcgaccagctgctgaccttgaaccagcacgtggagcagctgcgcggcgccttcgaccagacagtgtccctggccgtgggcttcattctgggcagcgcggccgccgagcgaggggtcctcagggacccgtgccagtga

SEQ ID NO: 6 - DNA, 55 bp, Artifical Sequence
  gene (1)..(55): DNA sequence for p53siRNA
tgactccagtggtaatctacttcaagagagtagattaccactggagtcttttttc

SEQ ID NO: 7 - DNA, 59 bp, Artifical Sequence
  gene (1)..(59): DNA sequence for p53siRNA
tcgagaaaaaagactccagtggtaatctactctcttgaagtagattaccactggagtca

SEQ ID NO: 8 - DNA, 57 bp, Artifical Sequence
  gene (1)..(57): DNA sequence for control siRNA
taattctccgaacgtgtcacgttcaagagacgtgacacgttcggagaattttttttc

SEQ ID NO: 9 - DNA, 61 bp, Artifical Sequence
  gene (1)..(61): DNA sequence for control siRNA
tcgagaaaaaaaattctccgaacgtgtcacgtctcttgaacgtgacacgttcggagaatta

SEQ ID NO: 10 - DNA, 7650 bp, Artifical Sequence
  gene (1)..(7650): pLL3.7 vector
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgttaacgcgcggtgaccctcgaggtcgacggtatcgataagctcgcttcacgagattccagcaggtcgagggacctaataacttcgtatagcatacattatacgaagttatattaagggttccaagcttaagcggccgcgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaggaattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 11 - DNA, 31 bp, Artifical Sequence
  misc_feature (1)..(31): KLF4-sense primer
tactcgaggccaccatggctgtcagcgacgc

SEQ ID NO: 12 - DNA, 31 bp, Artifical Sequence
  misc_feature (1)..(31): KLF4-antisense primer
ggcgaattcattaaaaatgcctcttcatgtg

SEQ ID NO: 13 - DNA, 30 bp, Artifical Sequence
  misc_feature (1)..(30): SOX2-sense primer
cctcgagccaccatgtacaacatgatggag

SEQ ID NO: 14 - DNA, 28 bp, Artifical Sequence
  misc_feature (1)..(28): SOX2-antisense primer
cggaattcatcacatgtgtgagaggggc

SEQ ID NO: 15 - DNA, 30 bp, Artifical Sequence
  misc_feature (1)..(30): CMYC-sense primer
actcgagccaccatgcccctcaacgttagc

SEQ ID NO: 16 - DNA, 30 bp, Artifical Sequence
  misc_feature (1)..(30): CMYC-antisense primer
cggaattcattacgcacaagagttccgtag

SEQ ID NO: 17 - DNA, 30 bp, Artifical Sequence
  misc_feature (1)..(30): Oct4-sense primer
aggatccgccaccatggcgggacacctggc

SEQ ID NO: 18 - DNA, 30 bp, Artifical Sequence
  misc_feature (1)..(30): Oct4-antisense primer
gcgaattcatcagtttgaatgcatgggagg

SEQ ID NO: 19 - DNA, 31 bp, Artifical Sequence
  misc_feature: UTF1-sense primer
aactcgagccaccatgctgctccggccccgc

SEQ ID NO: 20 - DNA, 30 bp, Artifical Sequence
  misc_feature (1)..(30): UTF1-antisense primer
aagaattccactggcacgggtccctgagga

SEQ ID NO: 21 - DNA, 20 bp, Artifical Sequence
  misc_feature (1)..(20): Oct4 forward primer
gaaccgagtgagaggcaacc

SEQ ID NO: 22 - DNA, 20 bp, Arifical Sequence
  misc_feature (1)..(20): Oct4 reverse primer
atcccaaaaaccctggcaca

SEQ ID NO: 23 - DNA, 20 bp, Artifical Sequence
  misc_feature (1)..(20): SOX2 forward primer
atgggttcggtggtcaagtc

SEQ ID NO: 24 - DNA, 20 bp, Artifical Sequence
  misc_feature (1)..(20): SOX2 reverse primer
ccctcccatttccctcgttt

SEQ ID NO: 25 - DNA, 20 bp, Artifical Sequence
  misc_feature (1)..(20): c-MYC forward primer
ggaacaagaagatgaggaag

SEQ ID NO: 26 - DNA, 19 bp, Artifical Sequence
  misc_feature (1)..(19): c-MYC reverse primer
tgattgctcaggacatttc

SEQ ID NO: 27 - DNA, 20 bp, Artifical Sequence
  misc_feature (1)..(20): KLF4-forward primer
gcaaaacctacacaaagagt

SEQ ID NO: 28 - DNA, 20 bp, Artifical Sequence
  misc_feature (1)..(20): KLF4-reverse primer
gaccatgattgtagtgcttt

SEQ ID NO: 29 - DNA, 19 bp, Artifical Sequence
  misc_feature (1)..(19): OCT4-Tg
gaaggatgtggtccgagtg

SEQ ID NO: 30 - DNA, 18 bp, Artifical Sequence
  misc_feature (1)..(18): SOX2-Tg
catgggttcggtggtcaa

SEQ ID NO: 31 - DNA, 20 bp, Artifical Sequence
  misc_feature (1)..(20): c-MYC-Tg
tacatcctgtccgtccaagc

SEQ ID NO: 32 - DNA, 18 bp, Artifical Sequence
  misc_feature (1)..(18): KLF4-Tg
accactgtgactgggacg

SEQ ID NO: 33 - DNA, 20 bp, Artifical Sequence
  misc_feature (1)..(20): pLl-Tg
gcagcgtatccacatagcgt

SEQ ID NO: 34 - DNA, 19 bp, Artifical Sequence
  misc_feature (1)..(19): Oct4 forward primer
attcagccaaacgaccatc

SEQ ID NO: 35 - DNA, 19 bp, Artifical Sequence
  misc_feature (1)..(19): Oct4 reverse primer
ggaaagggaccgaggagta

SEQ ID NO: 36 - DNA, 18 bp, Artifical Sequence
  misc_feature (1)..(18): SOX2 forward primer
cagcgcatggacagttac

SEQ ID NO: 37 - DNA, 18 bp, Artifical Sequence
  misc_feature (1)..(18): SOX2 reverse primer
ggagtgggaggaagaggt

SEQ ID NO: 38 - DNA, 18 bp, Artifical Sequence
  misc_feature (1)..(18): c-MYC forward primer
agtttcatctgcgacccg

SEQ ID NO: 39 - DNA, 21 bp, Artifical Sequence
  misc_feature (1)..(21): c-MYC reverse primer
cctcatcttcttgttcctcct

SEQ ID NO: 40 - DNA, 18 bp, Artifical Sequence
  misc_feature (1)..(18): KLF4-Forward primer
gcgggaagggagaagaca

SEQ ID NO: 41 - DNA, 18 bp, Artifical Sequence
  misc_feature (1)..(18): KLF4 reverse primer
ccggatcggataggtgaa

SEQ ID NO: 42 - DNA, 18 bp, Artifical Sequence
  misc_feature (1)..(18): REX1 forward primer
cctaaacagctcgcagaa

SEQ ID NO: 43 - DNA, 18 bp, Artifical Sequence
  misc_feature (1)..(18): REX1 reverse primer
cagccttgaaagggacac

SEQ ID NO: 44 - DNA, 18 bp, Artifical Sequence
  misc_feature (1)..(18): LIN28 forward primer
gggcatctgtaagtggtt

SEQ ID NO: 45 - DNA, 18 bp, Artifical Sequence
  misc_feature (1)..(18): LIN28 reverse primer
gtagggctgtggatttct

SEQ ID NO: 46 - DNA, 18 bp, Artifical Sequence
  misc_feature (1)..(18): SALL4 forward primer
tgatgggagaccaggagt

SEQ ID NO: 47 - DNA, 18 bp, Artifical Sequence
  misc_feature (1)..(18): SALL4 reverse primer
gcgggctgagttattgtt

SEQ ID NO: 48 - DNA, 19 bp, Artifical Sequence
  misc_feature (1)..(19): NODAL forward primer
acatcatccgcagcctaca

SEQ ID NO: 49 - DNA, 19 bp, Artifical Sequence
  misc_feature (1)..(19): NODAL reverse primer
ccatctgaaaccgctctaa

SEQ ID NO: 50 - DNA, 18 bp, Artifical Sequence
  misc_feature (1)..(18): TDGF1 forward primer
tcaggaatttgctcgtcc

SEQ ID NO: 51 - DNA, 17 bp, Artifical Sequence
  misc_feature (1)..(17): TDGF1 reverse primer
cttgggcagccaggtgt

SEQ ID NO: 52 - DNA, 19 bp, Artifical Sequence
  misc_feature (1)..(19): OTX2 forward primer
ccacttcgggtatggactt

SEQ ID NO: 53 - DNA, 19 bp, Artifical Sequence
  misc_feature (1)..(19): OTX2 reverse primer
ttgttggcggcacttagct

SEQ ID NO: 54 - DNA, 18 bp, Artifical Sequence
  misc_feature (1)..(18): ZNF206 forward primer
tggagtgcctgacctttg

SEQ ID NO: 55 - DNA, 18 bp, Artifical Sequence
  misc_feature (1)..(18): ZNF206 reverse primer
cgcatgtgcagcttgaga

SEQ ID NO: 56 - DNA, 18 bp, Artifical Sequence
  misc_feature (1)..(18): LEFTY1 forward primer
caaccgcacctccctcat

SEQ ID NO: 57 - DNA, 18 bp, Artifical Sequence
  misc_feature (1)..(18): LEFTY1 reverse primer
ttctcggcccacttcatc

SEQ ID NO: 58 - DNA, 18 bp, Artifical Sequence
  misc_feature (1)..(18): LEFTY2 forward primer
caaccgcacctccctcat

SEQ ID NO: 59 - DNA, 19 bp, Artifical Sequence
  misc_feature (1)..(19): LEFTY2 reverse primer
cagttcttggcccacttca

SEQ ID NO: 60 - DNA, 18 bp, Artifical Sequence
  misc_feature (1)..(18): DPPA4 forward primer
ccaatccctccattacct

SEQ ID NO: 61 - DNA, 18 bp, Artifical Sequence
  misc_feature (1)..(18): DPPA4 reverse primer
ccctggctgaaattctcg

SEQ ID NO: 62 - DNA, 18 bp, Artifical Sequence
  misc_feature (1)..(18): SALL2 forward primer
ggcgaaagcagcggaaac

SEQ ID NO: 63 - DNA, 18 bp, Artifical Sequence
  misc_feature (1)..(18): SALL2 reverse primer
tgtggcagcgaccaggaa

SEQ ID NO: 64 - DNA, 18 bp, Artifical Sequence
  misc_feature (1)..(18): NANOG forward primer
tgcctcacacggagactg

SEQ ID NO: 65 - DNA, 18 bp, Artifical Sequence
  misc_feature (1)..(18): NANOG reverse primer
gctattcttcggccagtt

SEQ ID NO: 66 - DNA, 21 bp, Artifical Sequence
  misc_feature (1)..(21): DNMT3B forward primer
gacgatggctatcagtcttac

SEQ ID NO: 67 - DNA, 20 bp, Artifical Sequence
  misc_feature (1)..(20): DNMT3B reverse primer
cctacctttatgcccaactc

SEQ ID NO: 68 - DNA, 23 bp, Artifical Sequence
  misc_feature (1)..(23): GATA2 forward primer
ccacccaaagaagtgtctcctga

SEQ ID NO: 69 - DNA, 21 bp, Artifical Sequence
  misc_feature (1)..(21): GATA2 reverse primer
cggttctgcccattcatcttg

SEQ ID NO: 70 - DNA, 21 bp, Artifical Sequence
  misc_feature (1)..(21): SCL forward primer
tctctcggcagcgggttcttt

SEQ ID NO: 71 - DNA, 22 bp, Artifical Sequence
  misc_feature (1)..(22): SCL reverse primer
ccaggcggaggatctcattctt

SEQ ID NO: 72 - DNA, 21 bp, Artifical Sequence
  misc_feature (1)..(21): CD31 forward primer
ggtgacactggacaagaaaga

SEQ ID NO: 73 - DNA, 20 bp, Artifical Sequence
  misc_feature (1)..(20): CD31 reverse primer
gacatcggaaggataaaacg

SEQ ID NO: 74 - DNA, 20 bp, Artifical Sequence
  misc_feature (1)..(20): KDR forward primer
aggggcacgattccgtcaag

SEQ ID NO: 75 - DNA, 20 bp, Artifical Sequence
  misc_feature (1)..(20): KDR reverse primer
tttcaaagggaggcgagcat

SEQ ID NO: 76 - DNA, 21 bp, Artifical Sequence
  misc_feature (1)..(21): Oligo2 forward primer
gcatctgcgaacccaagcaat

SEQ ID NO: 77 - DNA, 21 bp, Artifical Sequence
  misc_feature (1)..(21): Oligo2 reverse primer
acccgccaagaaaggaggaag

SEQ ID NO: 78 - DNA, 24 bp, Artifical Sequence
  misc_feature (1)..(24): RUNX1 forward primer
tgtggtcctatttaagccagcccc

SEQ ID NO: 79 - DNA, 24 bp, Artifical Sequence
  misc_feature (1)..(24): RUNX1 reverse primer
tcaggctgggcacgacgaatgctc

SEQ ID NO: 80 - DNA, 18 bp, Artifical Sequence
  misc_feature (1)..(18): AFP forward primer
aaatgcgtttctcgttgc

SEQ ID NO: 81 - DNA, 20 bp, Artifical Sequence
  misc_feature (1)..(20): AFP reverse primer
cagcctcaagttgttcctct

SEQ ID NO: 82 - DNA, 23 bp, Artifical Sequence
  misc_feature (1)..(23): CK19 forward primer
accaagtttgagacggaacaggc

SEQ ID NO: 83 - DNA, 23 bp, Artifical Sequence
  misc_feature (1)..(23): CK19 reverse primer
tgtcttccaaggcagctttcatg

SEQ ID NO: 84 - DNA, 27 bp, Artifical Sequence
  misc_feature (1)..(27): OCT4-4 forward primer
ggatgttattaagatgaagatagttgg

SEQ ID NO: 85 - DNA, 25 bp, Artifical Sequence
  misc_feature (1)..(25): OCT4-4 reverse primer
cctaaactccccttcaaaatctatt

SEQ ID NO: 86 - DNA, 24 bp, Artifical Sequence
  misc_feature (1)..(24): OCT4-6 forward primer
gaaggggaagtagggattaatttt

SEQ ID NO: 87 - DNA, 25 bp, Artifical Sequence
  misc_feature (1)..(25): OCT4-6 reverse primer
caacaaccataaacacaataaccaa

SEQ ID NO: 88 - DNA, 23 bp, Artifical Sequence
  misc_feature (1)..(23): OCT4-8 forward primer
aagtttttgtgggggatttgtat

SEQ ID NO: 89 - DNA, 23 bp, Artifical Sequence
  misc_feature (1)..(23): OCT4-8 reverse primer
ccacccactaaccttaacctcta

SEQ ID NO: 90 - DNA, 30 bp, Artifical Sequence
  misc_feature (1)..(30): NANOG-2 forward primer
gagttaaagagttttgtttttaaaaattat

SEQ ID NO: 91 - DNA, 30 bp, Artifical Sequence
  misc_feature (1)..(30): NANOG-2 reverse primer
tcccaaatctaataatttatcatatctttc